(19) United States
(12) Patent Application Publication
(10) Pub. No.: US 2014/0296174 A1
(43) Pub. Date: Oct. 2, 2014

(54) SILK-BASED MULTIFUNCTIONAL BIOMEDICAL PLATFORM

(71) Applicant: TUFTS UNIVERSITY, Medford, MA (US)

(72) Inventors: Fiorenzo Omenetto, Wakefild, MA (US); David L. Kaplan, Concord, MA (US)

(21) Appl. No.: 14/351,047

(22) PCT Filed: Oct. 11, 2012
(86) PCT No.: PCT/US2012/059834

§ 371 Date: Apr. 10, 2014

Related U.S. Application Data
(60) Provisional application No. 61/545,786, filed on Oct. 11, 2011.

(51) Int. Cl. A61K 47/42; A61K 31/704

(52) U.S. Cl. CPC A61K 47/42; A61K 31/704
USPC 514/34; 514/773

(57) ABSTRACT

The present invention provides a multifunctional platform suitable for biomedical applications. In particular, the invention includes a silk fibroin-based multifunctional device that enables both the sustained delivery of a drug and monitoring of such delivery in vivo.

### RELATED APPLICATIONS

This International application claims the benefit of and priority to U.S. Provisional Application Ser. No. 61/545,786, entitled “SILK-BASED DELIVERY SYSTEM FOR THE LOCAL APPLICATION OF DOXORUBICIN” filed Oct. 11, 2011, the contents of which are incorporated herein by reference in its entirety.

### GOVERNMENT SUPPORT

The work presented in this application was supported in part by the U.S. Army Research Laboratory and the U.S. Army Research Office under contract number W911 NF-07-1-0618 and by the DARPA-DSO, the AFOSR and the NIH P41 Tissue Engineering Resource Center. SEM images were obtained at the Center for Nanoscale Systems (CNS), a member of the National Nanotechnology Infrastructure Network (NNIN), which is supported by the National Science Foundation under NSF award number ECS-0335765. The United States government has certain rights in the invention.

### BACKGROUND

Silk is a natural protein fiber produced in a specialized gland of certain organisms, such as the silkworm, Bombyx mori, certain spiders (e.g., spider silk), Hymenoptera (bees, wasps, and ants), etc. From a materials science perspective, silks spun by spiders and silkworms represent the strongest and toughest natural fibers known. Over five millennia of history accompany the journey of silk from a sought-after textile to a scientifically attractive fiber. More recently, the novel material features of silks have been extended due to insights into self-assembly and the role of water in assembly. These insights, in turn, have led to new processing methods to generate silk-based hydrogels, ultrathin films, thick films, conformal coatings, three-dimensional porous matrices, solid blocks, nanoscale diameter fibers, and large diameter fibers. In addition to its structural attributes, such as durability, elasticity and flexibility, silk also is noteworthy for its biocompatibility. Biocompatibility broadly refers to silk's safe and non-toxic nature, including being biodegradable, bioresorbable, and non-antigenic (e.g., does not cause irritation or induce immune response).

Unique physiochemical properties of silk have led to the possibility that silk-based materials may be suitable for a variety of biomedical applications. However, implementation of silk-based devices in biological contexts has so far been limited for a number of technical challenges.

### SUMMARY OF THE INVENTION

The present invention provides a multifunctional platform suitable for in vivo clinical applications with remarkable attributes.

Among other things, the invention provides a multifunctional optical device that combines the ability to (i) stabilize a drug in a delivery matrix, (ii) deliver a drug over time (e.g., sustained drug release) and the ability to (iii) monitor such delivery in situ by measuring structural changes of the device itself that directly correlate with the delivery of the agent. In some embodiments additional functionality may also be incorporated.

Such multifunctional devices may be fabricated from a biocompatible and bioresorbable material, such as silk fibroin-based matrix. According to the present invention, one or more desirable agents, such as drugs, can be incorporated into a silk fibroin-based material to form a silk fibroin delivery matrix, which can function as a drug delivery device with desirable optical features. Thus, in some embodiments, the invention provides implantable, multifunctional bioresorbable optics that comprise a silk fibroin-based matrix.

An exemplary delivery device described herein comprises a silk fibroin matrix and a biologically active agent incorporated therein. In some embodiments, a delivery device comprising a silk fibroin matrix is fabricated in such a way that it has certain optical features, which can be detected and measured by suitable optical measurements. This provides the ability to monitor drug release in vivo by detecting changes in optical properties of the silk fibroin matrix. As described in more detail herein, the structural integrity of the silk fibroin matrix itself directly correlates with the amount of drug release that occurs as the matrix degrades at a controlled rate and releases the drug.

In some embodiments, a silk fibroin matrix with a drug incorporated therein is placed (e.g., implanted) at a desirable site in a subject, where the drug is released from the delivery matrix over time for in vivo administration. The release of the drug can be subsequently monitored in vivo by optical measurements of the silk fibroin matrix. Drug release occurs as the silk fibroin delivery matrix that contains the drug degrades over time in vivo. Such degradation of the delivery matrix coincides structural changes of the device, which result in measurable changes in optical features/parameters of the silk fibroin matrix itself. Thus, change in certain optical parameters of the silk delivery device is directly correlated with the amount of the drug released in vivo.

More specifically, The present invention encompasses the recognition that prism devices as described for example in WO 2011/046652 A2 (PCT/US2010/42585) can be beneficially utilized to achieve controlled and/or monitored release of agents from the prism device. Among other things, the present invention teaches that behavior of certain such prism devices can provide information about extent/degree and/or nature of release of agents from the devices. The present invention harnesses this insight and provides systems for capturing and/or acting upon such information.

For example, the present invention teaches that release of agent from a prism device can result in structural changes in the device that can be detected and that embody information about the release. The present invention provides systems for capturing such information, and therefore for monitoring release. The present invention further provides systems for controlling or modifying release that involve capturing such information in order to modify release and then making appropriate adjustments as desired.

To date, there is no platform available in the art for sustained drug release which allows correlates to monitoring the process in situ related to the amount of drug that has been released. It is contemplated that a possible scenario for practical applications of such a device includes the following: for a patient with cancer where the lymph nodes were removed, the surgical site may be coated with an ad-hoc silk mirror that will provide sustained release of a chemotherapeutic drug; subsequently, the amount of chemo delivery can be monitored through optical imaging per the optical transduction demonstrated herein. Further, the device has the option of co-doping with other constituents such as NP to control tumor margins through photo-/thermal-therapy. Finally, once the job is finished, there is no need for a second surgery as the device will fully resorb over time in vivo.

### BRIEF DESCRIPTION OF THE DRAWING

FIG. 1 depicts the chemical structure of doxorubicin HCl.

FIG. 2 provides graphs showing cumulative in vitro release of doxorubicin from a silk fibroin delivery matrix over a period of 20 days.

FIG. 3 provides a graph showing cumulative doxorubicin release from silk films relative to varied proteolytic degradation. N=3, error bars represent standard deviations (where error bars absent, they fall into the plot symbol). Doxorubicin quantified via absorbance at 495 nm (limit of detection=approx. 4 μg/mL).

FIG. 4 provides mages of doxorubicin stored for 3 weeks in silk films or in solution at −20° C. and 60° C. with fluorescent doxorubicin remaining in the various storage states (0.8 mg/mL and 7 mg/mL in solution, encapsulated in silk films) after 3 weeks at −20° C. and 60° C. N=3, error bars represent standard deviations).

FIG. 5 provides graphs showing effects of doxorubicin with or without silk fibroin delivery matrix on tumor growth: (A) Doxrobicin loaded silk films reduced primary tumour growth in vivo. MDA-MB-231 human breast tumours were injected orthotopically in NOD/SCID mice. Following tumour formation mice were treated with either doxorubicin loaded silk films or with the equivalent amount of doxorubicin i.v. and primary tumour weight was determined at the end of the study; (B) Impact of doxrobicin loaded silk films on primary tumour growth in vivo. MDA-MB-231 human breast tumours were injected orthotopically in NOD/SCID mice. Following tumour formation mice were treated with either doxorubicin loaded silk films or with the equivalent amount of doxorubicin i.v.

FIG. 6 provides graphs showing drug release from silk matrices: (A) Cumulative in vitro release of doxorubicin from silk hydrogels over 28 days. N=3, error bars represent standard deviation, where error bars are not shown they are within the plot symbol; (B) Cumulative in vitro release of doxorubicin from silk hydrogels over 28 days. N=3, error bars represent standard deviation, where error bars are not shown they fade into background.

FIG. 7 provides graphs showing cell killing effects of doxorubicin with or without silk fibroin delivery matrix. Cell viability for cultures exposed to various treatment groups for 72 h.

FIG. 8 illustrates silk fibroin platform comprising microprisms: (a) Scanning electron microscope image of a silk micro-prism array (MPA). (b) The schematic of the experimental setup for the evaluation of the performance of a replicated MPA. Incoherent white light illumination was provided to the silk reflector from a fixed height and a backscattering reflection probe is used to collect the response from the same height and couple it to a spectrometer. (c) The silk MPA shows significant increase in reflected signal compared to the unpatterned plain film. (d) & (e) Results from in vitro experiments from tissue layers characterized with the setup shown in (b), where a silk MPA is placed underneath a spectrally responsive element, a layer of cellulose embedded with red pigment, to capture scattered photons in the forward direction and enhanced the backscattered signal. (d) Comparison between the signal detected from (1) the spectral element covered by one layer of fat, (2) the same covered by two fat layers and (3) the same as in (2) but with the silk MPA under the spectral element. The reflectivity response was significantly higher when the mirror was present.

The same experiment was repeated by using layers of muscle tissue. The data are presented in (e) that compares (1) the response due to the spectral element covered with two layers of muscle tissue with (2) the same with the reflector in place. The arrows indicate the absorption peaks of the tissue.

FIG. 9 provides schematics and experimental data summarizing mirror performance at deeper depth. Phantom results for demonstrating signal and contrast enhancement with the MPAs in deep tissue. (a) The schematic of the experimental setup with a variable source detector separation for imaging deeper layers. Illumination was provided to the phantom, with the fiber tip touching the phantom surface. The detection fiber is scanned along the phantom and the reflector is placed in a depth of 1 cm. The detection fiber was coupled it to a spectrometer. (b) The MPA shows significant increase in reflected signal compared to measuring reflectance from the phantom alone. (c) This increase in signal reduces with larger source detector distances. (d) Scanning geometry for contrast imaging. For contrast measurements, a 8 mm×8 mm ND filter is additionally put on top of the reflector, mimicking a local inclusion. (e) The contrast enhancement for measuring the ND filter is increased 3.5 times at source detector distance of 12 mm and also decreases with larger separations (f), still showing a 2 times increase at 20 mm.

FIG. 10 provides images and data obtained from implanted silk fibroin microprisms. In vivo results from the use of silk MPAs and Au-NP MPAs. (a) The MPAs for implantation were prepared with a size of ˜1 cm×1 cm. (b) shows the subcutaneous implantation of a silk MPA in the dorsal region of a mouse. (c) The backscattered signal was measured in-vivo and showing ˜3× enhancement due to the MPA right after implantation. (d) Au-NPs doped silk MPAs with dimensions of approximately 1 cm×1 cm were prepared for implantation. (e) The Au-NP-silk solution, which was used to cast MPAs show enhanced absorption due to the Au-NP doping, as illustrated. (f) The backscattered signal of the implanted silk MPA was measured and compared to a control signal taken from an Au-NPs doped flat (i.e., unpatterned) silk film also implanted in the mouse's dorsal region as a control. The measurement shows similar signal enhancement as the undoped counterparts of FIG. 10C.

FIG. 11 illustrates a multifunctional optical device employed for therapeutic application of silk fibroin delivery device containing a drug and changes in reflectivity of the device over time. Chemotherapeutic (e.g., doxorubicin) loaded silk reflectors (DxRMPAs) are characterized in vitro under enzymatic degradation (proteinase k with a concentration of 0.1 mg/mL). Figure (a) illustrates the drug release (top) and the reflectivity of the DxR-MPAs (bottom) which also shows in the inset the optical microscope image of a portion of the DxR doped micro-prisms. The data is collected during the burst release (hourly up to 6 hours) and during the sustained release phase (every 6 hours, up to 30 hours) (N=6). (b) SEM images of DxR-MPA structures at 0 hour, 6 hours, and 30 hours, showing a gradual breakdown of the micro-prisms due to the enzymatic degradation (Scale bar: 50 μm). (c) A comparison between DxR dissolved in ultrapure water and doxorubicin-loaded silk MPAs (both with a concentration of 0.8 mg/mL) after storage at three weeks at −20° C. and 60° C. for 3 weeks. The silk DxR MPA maintains the fluorescence of the DxR after 3 weeks at 60° C. in contrast to the solution. (d) The DxR-MPAs were then fully degraded with 10 mg/mL proteinase k solution and were compared with the DxR solution by measuring the fluorescence intensity (excitation=430 nm, emission=550 nm) to determine the chemical activity of the drug. DxR fluorescence decreases when stored in solution, while the fluorescence of the DxR stored in MPAs does not significantly decrease with the 80° C. increase in storage temperature (two-tailed P value p<0.02 at −20° C., and p<0.001 at 60° C., Student t-test).

FIG. 12 provides microscopy images of the silk micro-prism reflectors used for the implants.

FIG. 13 provides SEM images of silk MPAs.

FIG. 14 (a) illustrates enzymatic degradation of silk films by protease XIV under standard reaction conditions at 37° C. The temperatures on the right side of the Figure indicate the temperatures used to anneal the films to control crystalline content (at 4° C., 25° C., 37° C., 70° C., 95° C.); (b) FTIR measured absorbance spectra of differently treated silk films in terms of annealing duration, showing distinct peaks which correspond to different silk protein structures and degradation/dissolution times.

FIG. 15 provides a silk micro-prism reflector showing several orders of magnitude increase in reflected signal compared to the background signal in open air when the signal is collected at a distance from the sample. (1) is the reflected background from a diffusing surface whereas (2) is the reflected signal at the same distance with a microprism located onto the diffusing surface.

FIG. 16 shows a schematic setup and images of silk micro-prism reflectors embedded in scattering media. The system was exposed to isotropic illumination from a white light source—the reflection from the films was collected at a distance of 1.5 meters with a digital CCD camera as illustrated in (a). Figure (b) shows images of the silk reflector and a flat substrate under 3.5 cm of gelatin and the corresponding lineout extracted from the image. Figure (c) shows the image acquired from the CCD when the silk reflector was immersed under 6.5 cm of scattering solution composed of talcum and water. The silk reflector was attached to the bottom of a dark container and then covered with the solution.

FIG. 17 illustrates a model imaging set-up for a silk fibroin matrix optical device.

FIG. 18 Spectral response of both the pigmented cellulose layer and the multilayered spectral filter used in the experiments. The layout of the experiment is illustrated at the top and serves as the basis for the layout of the in-vitro experiment.

FIG. 19 Results from in-vitro experiments measuring the baseline absorbance and corresponding reflection of a single layer of porcine muscle tissue. Two absorption features are detectable at wavelengths of ˜550 nm and ˜575 nm. These features are apparent in the MPA enhanced measurement described in the main text.

FIG. 20 Results from in-vitro experiments with incoherent illumination and detection through a fiber probe of the backscattered spectrum through porcine muscle tissue layers in the presence of a multilayer (λ0=600 nm, Δλ˜10 nm) dielectric notch filter and resulting enhancement of the detected response with the addition of a silk micro-prism array. The figure also shows the bandwidth of the dielectric notch filter superimposed on the reflection baseline measurement shown in FIG. 19.

FIG. 21 Results from in-vitro deep tissue experiments. The MPA shows significant increase in reflected signal compared to measuring reflectance from the phantom alone. This increase in signal is slightly reduced in the liquid phantom (without ink) and further reduced with ink as an absorbing material. A 15-20% enhancement could still be found at 10 mm depth in the liquid absorbing phantom.

FIG. 22 Results from in-vitro deep tissue experiments. The contrast enhancement for measuring the ND filter is increased 3.5 times at source detector distance of 12 mm and is still 3 times for the liquid phantom without ink. The contrast increase in the absorbing liquid phantom was 1.5 times at the same source detector distance and even bigger for larger source detector distances.

FIG. 23 Reflectivity spectra of undoped micro-prism film and plain silk at (a) 0 week and (b) 2 weeks (N=3). The optical fiber probe was placed against the mouse skin for the reflectivity measurements. The integration time was 25 millisecond with data average number=10.

FIG. 24 Reflectivity spectra of micro-prism film, plain silk and bare mouse skin. The optical fiber probe was placed against the mouse skin for the reflectivity measurements. The optical fiber probe was placed against the mouse skin for the reflectivity measurements. The integration time was 25 millisecond with data average number=10.

FIG. 25 (a) Schematic of the geometry used for the simulation. The simulation models for the reflectivity (b) without reflectors and (c) with reflectors. (d) Comparison of temporal point spread function (TPSF) in the situations with and without reflector. Note that the peak of the maroon curve—i.e. with reflector—occurs around 6 ps, which is the “round trip” time for a photon to hit the microarray cube and be detected. The green curve—i.e. without reflector—is obtained in the tissue without microarray. It has been smoothed for display. Note that both curves share the same peak (very broad) around 2 ps, which is due to those photons that are detected without being reflected by the microarray. The speed of light in the tissue is assumed v=0.214 mm/ps.

FIG. 26 Post implant analysis of a silk film implanted in the dorsal region of a Balb/c mouse after 4 weeks of in-dwelling time. The arrows associated to (a), (b) and (c) provide some initial evidence of film reintegration and revascularization occurring around the implanted film.

FIG. 27 Full darkfield microscopy image of histological cross section of the 1 cm reflector implanted in the mouse (after 4 weeks of implantation time). Visible are the outer epidermis layer (1) and subcutaneous tissue (2), the silk film, the subcutaneous tissue (3, 4), and muscle tissue (6). The Subcutaneous tissue shows a thickening of the hypodermis directly under the implant (5) when compared to deeper hypodermis (3). The subcutaneous fat layer is unaffected (3).

FIG. 28 Reflectivity spectra of gold nanoparticle doped micro-prism film and plain silk at (a) 0 week and (b) 2 weeks (N=3). The optical fiber probe was placed against the mouse skin for the reflectivity measurements. The integration time was 25 millisecond with data average number=10.

FIG. 29 (a) Absorbance response of gold nanoparticles (Au-NPs) doped silk solution measured by UV-Vis spectrometer, showing a strong absorbance peak at ˜532 nm due to the plasmon resonant response of doped Au-NPs. Absorbance (b) and reflection (c) spectra of Au NPs doped silk film and reflector measured with Ocean Optics USB 2000 spectrometer.

FIG. 30 The doped MPAs show enhanced absorption due to the Au-nanoparticle doping, as illustrated in (c). This is used to inhibit bacterial growth because of local heating caused by enhanced absorption as shown in (d) which illustrates a Au-NP-MPA and an undoped MPA embedded in E. Coli lawn. A zone of bacteria inhibition was observed after the laser illumination around the Au-NPs MPA, in contrast to the undoped silk MPA under the same λ=532 nm illumination conditions.

FIG. 31 The Au-NP-silk solution, which was used to cast MPAs, show enhanced absorption due to the Au-NP doping, as illustrated in (a). This is used to inhibit bacterial growth because of local heating caused by enhanced absorption as shown in (b) which illustrates a Au-NP-MPA and an undoped MPA embedded in E. Coli lawn. A zone of bacteria inhibition was observed after the laser illumination around the Au-NPs MPA, in contrast to the undoped silk MPA under the same λ=532 nm illumination conditions. The same concept is applied in vivo using the Au-NPs MPA as a light-activated thermal patch as illustrated in the scheme (c). The results are shown in (d). A temperature increase of ˜5° C. at the implant site compared to surrounding tissues due to the Au-NPs absorption to green light is observed and recorded with a commercial IR camera (˜5 cm in diameter and with a power of ˜0.13 W/cm2). BOTTOM—image of a silk AuNP suture ribbon and its use to stitch an incision (and subsequent thermal image of the heating of the suture).

FIG. 32 Microscopy images of histological cross section of (a) the implanted Au-NP reflector and (b) Au-NP silk film after 2 weeks of implantation time.

FIG. 33 Doxorubicin concentration calibration curve by measuring the absorbance of the drug solution at 495 nm.

### DETAILED DESCRIPTION OF CERTAIN EMBODIMENTS

### Definitions

Administration: The term “administration” in the context of the present disclosure broadly refers to delivery of an agent, typically to a subject (e.g., a patient). Administration encompasses, for example, local administration, systemic administration, etc. As used herein, the term “systemic administration” refers to administration of an agent such that the agent becomes widely distributed in the body in significant amounts and has a biological effect, e.g., its desired effect, in the blood and/or reaches its desired site of action via the vascular system. Typical systemic routes of administration include administration by (1) introducing the agent directly into the vascular system or (2) oral, pulmonary, or intramuscular administration wherein the agent is adsorbed, enters the vascular system, and is carried to one or more desired site(s) of action via the blood. The phrase “local administration” on the other hand, refers to administration of an agent such that the agent is delivered locally and that the availability of the agent in the body is more concentrated at or near the region/site of effective delivery of the drug. Locally administered drugs may gradually diffuse to surrounding areas or tissues.

Agent: The terms “agent” and “agents” (as in “a therapeutic agent”) broadly encompasses any biologically active compounds or compositions, such as drugs. The terms “chemotherapeutic,” “anti-cancer agent” and “anti-cancer drug” may be used interchangeably. They refer to medications that are used to treat cancer or cancerous conditions. Anti-cancer drugs are conventionally classified in one of the following group: radioisotopes (e.g., Iodine-131, Lutetium-177, Rhenium-188, Yttrium-90), toxins (e.g., diphtheria, pseudomonas, ricin, gelonin), enzymes, enzymes to activate prodrugs, radio-sensitizing drugs, interfering RNAs, superantigens, anti-angiogenic agents, alkylating agents, purine antagonists, pyrimidine antagonists, plant alkaloids, intercalating antibiotics, aromatase inhibitors, anti-metabolites, mitotic inhibitors, growth factor inhibitors, cell cycle inhibitors, enzymes, topoisomerase inhibitors, biological response modifiers, anti-hormones and anti-androgens. Examples of anti-cancer agents include, but are not limited to, BCNU, cisplatin, gemcitabine, hydroxyurea, paclitaxel, temozolomide, topotecan, fluorouracil, vincristine, vinblastine, procarbazine, decarbazine, altretamine, methotrexate, mercaptopurine, thioguanine, fludarabine phosphate, cladribine, pentostatin, cytarabine, azacitidine, etoposide, teniposide, irinotecan, docetaxel, doxorubicin, daunorubicin, dactinomycin, idarubicin, plicamycin, mitomycin, bleomysin, tamoxifen, flutamide, leuprolide, goserelin, aminogluthimide, anastrozole, amsacrine, asparaginase, mitoxantrone, mitotane, and amifostine.

Effective amount: As used herein, the terms “effective amount” and “effective dose” refer to any amount or dose of a compound or composition that is sufficient to fulfill its intended purpose(s), i.e., a desired biological or medicinal response in a tissue or subject at an acceptable benefit/risk ratio. For example, in certain embodiments of the present invention, the purpose(s) may be: to inhibit angiogenesis, cause regression of neovasculature, interfere with activity of another bioactive molecule, cause regression of a tumor, inhibit metastases, reduce extent of metastases, etc. The relevant intended purpose may be objective (i.e., measurable by some test or marker) or subjective (i.e., subject gives an indication of or feels an effect). In some embodiments, a therapeutically effective amount is an amount that, when administered to a population of subjects that meet certain clinical criteria for a disease, disorder or condition (for example, as determined by symptoms manifested, disease progression/stage, genetic profile, etc.), a statistically significant therapeutic response is obtained among the population. A therapeutically effective amount is commonly administered in a dosing regimen that may comprise multiple unit doses. A therapeutically effective amount may be administered in a slow release, sustained delivery regimen. For any particular pharmaceutical agent, a therapeutically effective amount (and/or an appropriate unit dose within an effective dosing regimen) may vary, for example, depending on route of administration, on combination with other pharmaceutical agents. In some embodiments, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific pharmaceutical agent employed; the duration of the treatment; and like factors as is well known in the medical arts. In some embodiments, an effective amount is an amount that, when administered according to a particular regimen, produces a positive therapeutic outcome with a reasonably acceptable level of adverse side effects, such that the side effects, if present, are tolerable enough for a patient to continue with the therapeutic regimen. Those of ordinary skill in the art will appreciate that in some embodiments of the invention, a unit dosage may be considered to contain an effective amount if it contains an amount appropriate for administration in the context of a dosage regimen correlated with a positive outcome.

Inhibit: As used herein, the term “inhibit” means to prevent something from happening, to delay occurrence of something happening, and/or to reduce the extent or likelihood of something happening.

Local: The term “local” used in the context of “local delivery” or “local release” of an agent refers to a defined location at which a composition (e.g., a delivery device) described herein is positioned in vivo.

Localized lesion: As used herein, a “localized lesion” includes a site of diseased tissue, abnormal tissue, site of surgery, surgical cavity, resection sites, etc.

Preventing: As used herein, the term “preventing” when used to refer to the action of an agent to a process (e.g., metastasis, cancer progression, etc.) means reducing extent of and/or delaying onset of such a process when the agent (e.g., a therapeutic agent) is administered prior to development of one or more symptoms or attributes associated with the process.

Subject: The terms “subject” and “individual” are used herein interchangeably. They refer to a vertebrate, preferably human or another mammal (e.g., mouse, rat, rabbit, dog, cat, cattle, swine, sheep, horse or primate) that can be afflicted with or is susceptible to a disease, disorder or condition (e.g., cancer, injury, etc.) but may or may not have the disease, disorder or condition. In many embodiments, the subject is a human subject. In many embodiments, the subject is a patient. Unless otherwise stated, the terms “individual” and “subject” do not denote a particular age, and thus encompass adults, children, and newborn.

Susceptible: As used herein, the term “susceptible” means having an increased risk for and/or a propensity for (typically based on genetic predisposition, environmental factors, personal history, or combinations thereof) something, e.g., a disease, disorder, or condition (such as, for example, cancer) than is observed in the general population as a whole. The term takes into account that an individual “susceptible” for a condition may never be diagnosed with the condition.

Treat: As used herein, the term “treating” refers to partially or completely alleviating, ameliorating, relieving, delaying onset of, inhibiting progression of, reducing severity of, and/or reducing incidence of one or more symptoms or features of a particular disease, disorder, and/or condition. For example, “treating” a cancer may refer to inhibiting survival, growth, and/or spread of tumor cells; preventing, delaying, and/or reducing the likelihood of occurrence of metastases and/or recurrences; and/or reducing the number, growth rate, size, etc., of metastases. In some embodiments, “treating” also includes reducing unwanted or adverse side effects of drugs (e.g., therapeutics), without significantly compromising the effects of such drugs. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. In some embodiments, treatment comprises delivery of a pharmaceutical composition to a subject.

Silk fibroin matrix: As used herein, a “silk fibroin matrix” refers to a structural form made of a silk fibroin-based material. Typically, a silk-fibroin matrix is a solid-state composition, which can exist in a variety of forms. Examples of silk fibroin matrices include, without limitation, a film, a block, a thread, a gel, such as hydrogel, a powder, a particle, such as microspheres and nanospheres, and any combination thereof. Some embodiments of the invention comprise two or more portions of such matrices stuck together in layers and/or in blocks, each of which may be comprised of the same or different type of silk fibroin matrix materials. Depending on the type of compositions, silk fibroin matrices may be in a variety of forms, such as a free standing structure, a thin (virtually two-dimensional) forms with or without a substrate or support, particles that are dried or suspended in solution (i.e., a suspension), and so on. In some embodiments, a silk fibroin matrix is made substantially of purified silk fibroin. As used herein, “a silk fibroin delivery matrix” refers to a silk fibroin matrix that functions as a delivery device for an agent, such as pharmaceutical agents. Typically, an agent or agents desired to be delivered is incorporated into a silk fibroin matrix so as to form a silk fibroin delivery matrix. Methods for incorporating various agents into silk fibroin-based materials have previously been described.

### Multifunctional Platform

Advances in personalized medicine are symbiotic with the development of novel

technologies for biomedical devices. The present application presents an approach that combines diagnostics, therapeutics, and feedback about therapeutics in a single, implantable, biocompatible, and resorbable device. This confluence of form and function is accomplished by capitalizing on the unique properties of silk proteins as a mechanically robust, biocompatible, optically clear biomaterial matrix which can house, stabilize and retain the function of therapeutic components.

The present application provides data demonstrating that improved imaging for diagnostics and of treatment monitoring can be achieved by the use of a form of high-quality microstructured optical elements. The invention described herein introduces a novel platform for in vitro and in vivo use that provide functional biomaterials with built-in optical signal and contrast enhancement. The work presented herein demonstrates a family of devices that provides multi-functionality for simultaneous drug delivery and feedback about drug delivery with no adverse biological effects, all while slowly degrading, and in some cases aiding to regenerate native tissue.

The use of biocompatible materials is paramount for biomedical applications. Suitable biocompatible materials may be employed in a number of in vivo applications, such as structural supports, casings and implants, needed to integrate within the human body. Careful selection of such materials for each intended use is crucial to minimize risk of immune responses. Polymers such as polylactic acids and collagens have been widely studied as implantable, resorbable biomaterial matrices to fulfill a range of current or potential medical device needs.

More recently, the idea of integrating biocompatibility with technological functionality has emerged. Conceptually, the integration of preferred biological interface attributes of biomaterials with technological functionalities, such as electronics4 and optics5, provides a new and exciting path towards integrating devices within living tissue and at the same time eliminating the need for retrieval after their functional lifetimes are complete. In practice, however, enabling such a device poses a great deal of challenge. For example, for such an approach to be viable and operative, suitable biomaterial must meet the required material tolerances to favorably compare with common technical substrates, such as glass (e.g., silicon), plastics, and inorganic polymers. These requirements present a significant barrier to success as mechanical demands, optical clarity requirements and reliable electronic interfaces, among many other environmental impacts and insults, establish a range of material performance issues that are difficult to achieve. When further challenged by the needs for degradability and safety for tissue regeneration, the barriers to success only grow.

Recent results indicate that silk as a material possesses a convenient convergence of the individual features outlined above, suggesting a possibility that a path forward with this unique biomaterial. Silk is already a widely used, USDA-approved biopolymer and is not antigenic in humans (i.e., does not trigger adverse immune response). Further, silk has been shown to be suitable for use as a material platform for sophisticated optical and opto-electronic components with features on the micro- and nanoscale. The resulting free-standing devices formed from silk are refractive or diffractive, and comprise elements ranging from microlens arrays, white light holograms, to diffraction gratings and planar photonic crystals with minimum feature sizes of less than 20 nanometers. These components provide mechanically stable, high quality optical elements that are fully degradable, biocompatible and implantable. Additionally, silk materials have been shown to possess the ability to entrain and stabilize labile biological components which provides the opportunity for conceptualizing multifunctional devices, which may be suitable for in vivo use, such as for drug delivery and subsequent monitoring.

### Silk

Silk is a natural protein fiber produced in a specialized gland of certain organisms. Silk production is especially common in silkworms, as well as in the Hymenoptera (bees, wasps, and ants). Other types of arthropod also produce silk, most notably various arachnids such as spiders (e.g., spider silk). Silk fibers generated by insects and spiders represent the strongest natural fibers known and rival even synthetic high performance fibers.

Silk-based materials, e.g., those produced from silk fibroin, suitable for the present invention may be harvested from silk produced by a wide variety of species, including, without limitation: Antheraea mylitta; Antheraea pernyi; Antheraea yamamai; Galleria mellonella; Bombyx mori; Bombyx mandarina; Galleria mellonella; Nephila clavipes; Nephila senegalensis; Gasteracantha mammosa; Argiope aurantia; Araneus diadematus; Latrodectus geometricus; Araneus bicentenarius; Tetragnatha versicolor; Araneus ventricosus; Dolomedes tenebrosus; Euagrus chisoseus; Plectreurys tristis; Argiope trifasciata; and Nephila madagascariensis.

In some embodiments of the present invention, silk is produced by the silkworm, Bombyx mori.

In general, silk for use in accordance with the present invention may be produced by any such organism, or may be prepared through an artificial process, for example, involving genetic engineering of cells or organisms (e.g., transgenic and/or recombinant DNA technology) to produce a silk protein and/or chemical synthesis. In some embodiments, engineered silk fibroin may contain one or more mutations (see below for more detail).

As is known in the art, silks are modular in design, with large internal repeats flanked by shorter (˜100 amino acid) terminal domains (N and C termini) Silks have high molecular weight (200 to 350 kDa or higher) with transcripts of 10,000 base pairs and higher and >3000 amino acids (reviewed in Omenatto and Kaplan (2010) Science 329: 528-531). The larger modular domains are interrupted with relatively short spacers with hydrophobic charge groups in the case of silkworm silk. N- and C-termini are involved in the assembly and processing of silks, including pH control of assembly. The N- and C-termini are highly conserved, in spite of their relatively small size compared with the internal modules.

Table 1, below, provides an exemplary list of silk-producing species and silk proteins:

TABLE 1
An exemplary list of silk-producing species and silk proteins
(adopted from Bini et al. (2003), J. Mol. Biol. 335(1): 27-40).

| | | | |
|---|---|---|---|
| Accession | Species | Producing gland | Protein |

A. Silkworms

| | | | |
|---|---|---|---|
| AAN28165 | Antheraea mylitta | Salivary | Fibroin |
| AAC32606 | Antheraea pernyi | Salivary | Fibroin |
| AAK83145 | Antheraea yamamai | Salivary | Fibroin |
| AAG10393 | Galleria mellonella | Salivary | Heavy-chain |
| | | | fibroin |
| | | | (N-terminal) |
| AAG10394 | Galleria mellonella | Salivary | Heavy-chain |
| | | | fibroin |
| | | | (C-terminal) |
| P05790 | Bombyx mori | Salivary | Fibroin heavy |
| | | | chain precursor, |
| | | | Fib-H, H-fibroin |
| CAA27612 | Bombyx mandarina | Salivary | Fibroin |
| Q26427 | Galleria mellonella | Salivary | Fibroin light |
| | | | chain precursor, |
| | | | Fib-L, L-fibroin, |
| | | | PG-1 |
| P21828 | Bombyx mori | Salivary | Fibroin light |
| | | | chain precursor, |
| | | | Fib-L, L-fibroin |

B. Spiders

| | | | |
|---|---|---|---|
| P19837 | Nephila clavipes | Major ampullate | Spidroin 1, |
| | | | dragline silk |
| | | | fibroin 1 |
| P46804 | Nephila clavipes | Major ampullate | Spidroin 2, |
| | | | dragline silk |
| | | | fibroin 2 |
| AAK30609 | Nephila senegalensis | Major ampullate | Spidroin 2 |
| AAK30601 | Gasteracantha | Major ampullate | Spidroin 2 |
| | mammosa | | |
| AAK30592 | Argiope aurantia | Major ampullate | Spidroin 2 |
| AAC47011 | Araneus diadematus | Major ampullate | Fibroin-4, ADF-4 |
| AAK30604 | Latrodectus | Major ampullate | Spidroin 2 |
| | geometricus | | |
| AAC04503 | Araneus bicentenarius | Major ampullate | Spidroin 2 |
| AAK30615 | Tetragnatha versicolor | Major ampullate | Spidroin 1 |
| AAN85280 | Araneus ventricosus | Major ampullate | Dragline silk |
| | | | protein-1 |
| AAN85281 | Araneus ventricosus | Major ampullate | Dragline silk |
| | | | protein-2 |
| AAC14589 | Nephila clavipes | Minor ampullate | MiSp1 silk |
| | | | protein |
| AAK30598 | Dolomedes tenebrosus | Ampullate | Fibroin 1 |
| AAK30599 | Dolomedes tenebrosus | Ampullate | Fibroin 2 |
| AAK30600 | Euagrus chisoseus | Combined | Fibroin 1 |
| AAK30610 | Plectreurys tristis | Larger ampule- | Fibroin 1 |
| | | shaped | |
| AAK30611 | Plectreurys tristis | Larger ampule- | Fibroin 2 |
| | | shaped | |
| AAK30612 | Plectreurys tristis | Larger ampule- | Fibroin 3 |
| | | shaped | |
| AAK30613 | Plectreurys tristis | Larger ampule- | Fibroin 4 |
| | | shaped | |
| AAK30593 | Argiope trifasciata | Flagelliform | Silk protein |
| AAF36091 | Nephila | Flagelliform | Fibroin, |
| | madagascariensis | | silk protein |
| | | | (N-terminal) |
| AAF36092 | Nephila | Flagelliform | Silk protein |
| | madagascariensis | | (C-terminal) |
| AAC38846 | Nephila clavipes | Flagelliform | Fibroin, |
| | | | silk protein |
| | | | (N-terminal) |
| AAC38847 | Nephila clavipes | Flagelliform | Silk protein |
| | | | (C-terminal) |

### Silk Fibroin

Fibroin is a type of structural protein produced by certain spider and insect species that produce silk. Cocoon silk produced by the silkworm, Bombyx mori, is of particular interest because it offers low-cost, bulk-scale production suitable for a number of commercial applications, such as textile.

Silkworm cocoon silk contains two structural proteins, the fibroin heavy chain (˜350 k Da) and the fibroin light chain (˜25 k Da), which are associated with a family of non-structural proteins termed sericin, which glue the fibroin brins together in forming the cocoon. The heavy and light chains of fibroin are linked by a disulfide bond at the C-terminus of the two subunits (Takei, F., Kikuchi, Y., Kikuchi, A., Mizuno, S, and Shimura, K. (1987) J. Cell Biol., 105, 175-180; Tanaka, K., Mori, K. and Mizuno, S. (1993) J. Biochem. (Tokyo), 114, 1-4; Tanaka, K., Kajiyama, N., Ishikura, K., Waga, S., Kikuchi, A., Ohtomo, K., Takagi, T. and Mizuno, S. (1999) Biochim. Biophys. Acta, 1432, 92-103; Y Kikuchi, K Mori, S Suzuki, K Yamaguchi and S Mizuno, Structure of the Bombyx mori fibroin light-chain-encoding gene: upstream sequence elements common to the light and heavy chain, Gene 110 (1992), pp. 151-158). The sericins are a high molecular weight, soluble glycoprotein constituent of silk which gives the stickiness to the material. These glycoproteins are hydrophilic and can be easily removed from cocoons by boiling in water.

As used herein, the term “silk fibroin” refers to silk fibroin protein, whether produced by silkworm, spider, or other insect, or otherwise generated (Lucas et al., Adv. Protein Chem., 13: 107-242 (1958)). In some embodiments, silk fibroin is obtained from a solution containing a dissolved silkworm silk or spider silk. For example, in some embodiments, silkworm silk fibroins are obtained, from the cocoon of Bombyx mori. In some embodiments, spider silk fibroins are obtained, for example, from Nephila clavipes. In the alternative, in some embodiments, silk fibroins suitable for use in the invention are obtained from a solution containing a genetically engineered silk harvested from bacteria, yeast, mammalian cells, transgenic animals or transgenic plants. See, e.g., WO 97/08315 and U.S. Pat. No. 5,245,012, each of which is incorporated herein as reference in its entirety.

Thus, in some embodiments, a silk solution is used to fabricate compositions of the present invention contain fibroin proteins, essentially free of sericins. In some embodiments, silk solutions used to fabricate various compositions of the present invention contain the heavy chain of fibroin, but are essentially free of other proteins. In other embodiments, silk solutions used to fabricate various compositions of the present invention contain both the heavy and light chains of fibroin, but are essentially free of other proteins. In certain embodiments, silk solutions used to fabricate various compositions of the present invention comprise both a heavy and a light chain of silk fibroin; in some such embodiments, the heavy chain and the light chain of silk fibroin are linked via at least one disulfide bond. In some embodiments where the heavy and light chains of fibroin are present, they are linked via one, two, three or more disulfide bonds.

Although different species of silk-producing organisms, and different types of silk, have different amino acid compositions, various fibroin proteins share certain structural features. A general trend in silk fibroin structure is a sequence of amino acids that is characterized by usually alternating glycine and alanine, or alanine alone. Such configuration allows fibroin molecules to self-assemble into a beta-sheet conformation. These “Ala-rich” hydrophobic blocks are typically separated by segments of amino acids with bulky side-groups (e.g., hydrophilic spacers).

In some embodiments, core repeat sequences of the hydrophobic blocks of fibroin are represented by the following amino acid sequences and/or formulae: (GAGAGS)₅₋₁₅ (SEQ ID NO: 1); (GX)₅₋₁₅ (X=V, I, A) (SEQ ID NO: 2); GAAS (SEQ ID NO: 3); (S₁₋₂A₁₁₋₁₃) (SEQ ID NO: 4); GX₁₋₄ GGX (SEQ ID NO: 5); GGGX (X=A, S, Y, R, D V, W, R, D) (SEQ ID NO: 6); (S₁₋₂A₁₋₄)₁₋₂ (SEQ ID NO: 7); GLGGLG (SEQ ID NO: 8); GXGGXG (X=L, I, V, P) (SEQ ID NO: 9); GPX (X=L, Y, I); (GP(GGX)₁₋₄ Y)n (X=Y, V, S, A) (SEQ ID NO: 10); GRGGAn (SEQ ID NO: 11); GGXn (X=A, T, V, S); GAG(A)₆₋₇GGA (SEQ ID NO: 12); and GGX GX GXX (X=Q, Y, L, A, S, R) (SEQ ID NO: 13).

In some embodiments, a fibroin peptide contains multiple hydrophobic blocks, e.g., 3, 4, 5, 6, 7, 8, 9, 10, 11, 12, 13, 14, 15, 16, 17, 18, 19 and 20 hydrophobic blocks within the peptide. In some embodiments, a fibroin peptide contains between 4-17 hydrophobic blocks.

In some embodiments of the invention, a fibroin peptide comprises at least one hydrophilic spacer sequence (“hydrophilic block”) that is about 4-50 amino acids in length. Non-limiting examples of the hydrophilic spacer sequences include:

(SEQ ID NO: 14)
TGSSGFGPYVNGGYSG;
(SEQ ID NO: 15)
YEYAWSSE;
(SEQ ID NO: 16)
SDFGTGS;
(SEQ ID NO: 17)
RRAGYDR;
(SEQ ID NO: 18)
EVIVIDDR;
(SEQ ID NO: 19)
TTIIEDLDITIDGADGPI
and
(SEQ ID NO: 20)
TISEELTI.

In certain embodiments, a fibroin peptide contains a hydrophilic spacer sequence that is a derivative of any one of the representative spacer sequences listed above. Such derivatives are at least 75%, at least 80%, at least 85%, at least 90%, or at least 95% identical to any one of the hydrophilic spacer sequences.

In some embodiments, a fibroin peptide suitable for the present invention contains no spacer.

As noted, silks are fibrous proteins and are characterized by modular units linked together to form high molecular weight, highly repetitive proteins. These modular units or domains, each with specific amino acid sequences and chemistries, are thought to provide specific functions. For example, sequence motifs such as poly-alanine (polyA) and poly-alanine-glycine (poly-AG) are inclined to be beta-sheet-forming; GXX motifs contribute to 31-helix formation; GXG motifs provide stiffness; and, GPGXX (SEQ ID NO: 22) contributes to beta-spiral formation. These are examples of key components in various silk structures whose positioning and arrangement are intimately tied with the end material properties of silk-based materials (reviewed in Omenetto and Kaplan (2010) Science 329: 528-531).

It has been observed that the beta-sheets of fibroin proteins stack to form crystals, whereas the other segments form amorphous domains. It is the interplay between the hard crystalline segments, and the strained elastic semi amorphous regions, that gives silk its extraordinary properties. Non-limiting examples of repeat sequences and spacer sequences from various silk-producing species are provided in Table 2 below.

TABLE 2
Hydrophobic and hydrophilic components of fibroin sequences
(adopted from Bini et al. (2003), J. Mol. Biol. 335(1): 27-40).

| | Hydrophilic blocks | | | Hydrophobic blocks | | |
|---|---|---|---|---|---|---|
| | | | Hydrophilic | | | |
| | N- | C- | spacer (aa) & | | | Core |
| | term | term | representative | Range | # of | repeat |
| Species | aa | aa | sequence | (aa) | Blocks | sequences |

A. Lepidoptera (Heavy chain fibroin)

| | | | | | | |
|---|---|---|---|---|---|---|
| Bombyx | 151 | 50 | 32-33, | 159-607 | 12 | (GAGAGS)₅₋₁₅, (SEQ ID NO: 1); |
| mori | | | TGSSGFGPYVNGGYSG, | | | (GX)₅₋₁₅ (X=V, I, A), |
| | | | (SEQ ID NO: 14) | | | (SEQ ID NO: 2); |
| | | | | | | GAAS (SEQ ID NO: 3) |
| Bombyx | 151 | | YEYAWSSE, | | | |
| mandarina | | | (SEQ ID NO: 15) | | | |
| Antheraea | 86 | | SDFGTGS, | | | |
| mylitta | | | (SEQ ID NO: 16) | | | |
| Antheraea | 87 | 32 | | | | |
| pernyi | | | | | | |
| Antheraea | 87 | 32 | 7, | 140-340 | 16 | (S₁₋₂A₁₁₋₁₃), (SEQ ID NO: 4); |
| yamamai | | | RRAGYDR, | | | GX₁₋₄ GGX, (SEQ ID NO: 5); |
| | | | (SEQ ID NO: 17) | | | GGGX (X=A, S, Y, R, D V, |
| | | | | | | W, R, D), (SEQ ID NO: 6) |
| Galleria | 189 | 60 | 6-8, | 75-99 | 13 | (S₁₋₂A₁₋₄)₁₋₂, (SEQ ID NO: 7); |
| mellonella | | | EVIVIDDR, | | | GLGGLG, (SEQ ID NO: 8); |
| | | | (SEQ ID NO: 18) | | | GXGGXG (X=L, I, V, P), |
| | | | | | | (SEQ ID NO: 9); |
| | | | | | | GPX (X=L, Y, I) |

B. Arachnida

| | | | | | | |
|---|---|---|---|---|---|---|
| Nephila | 115 | 89 | | | | |
| Clavipes | | | | | | |
| Nephila | 115 | 89 | 26, | 260-380 | 5 | (GP(GGX)1-4 Y)n |
| madascariensis | | | TTIIEDLDITIDG ADGPI, | | | (X=Y, V, S, A), |
| | | | (SEQ ID NO: 19) | | | (SEQ ID NO: 10) |
| Argiope | | 113 | | | | GRGGAn, (SEQ ID NO: 11) |
| trifasciata | | | | | | GGXn (X=A, T, V, S) |
| Major | | | TISEELTI, | | | |
| ampullata | | | (SEQ ID NO: 20) | | | |
| Nephila | | 97 | No spacer | 19-46 | | GAG(A)₆₋₇GGA, |
| Clavipes | | | | | | (SEQ ID NO: 12); |
| | | | | | | GGX GX GXX(X=Q, Y, L, |
| | | | | | | A, S, R), (SEQ ID NO: 13) |
| Gasteracantha | | 89 | No spacer | | | |
| mammosa | | | | | | |
| Argiope | | 82 | No spacer | | | |
| aurantia | | | | | | |
| Nephila | | 82 | No spacer | | | |
| senegalensis | | | | | | |
| Latrodectus | | 88 | No spacer | | | |
| geometricus | | | | | | |
| Araneus | | 94 | No spacer | | | |
| diadematus | | | | | | |

The particular silk materials explicitly exemplified herein were typically prepared from material spun by silkworm, B. Mori. Typically, cocoons are boiled for ˜30 min in an aqueous solution of 0.02M Na₂CO₃, then rinsed thoroughly with water to extract the glue-like sericin proteins. The extracted silk is then dissolved in LiBr (such as 9.3 M) solution at room temperature, yielding a ˜20% (wt.) solution. The resulting silk fibroin solution can then be further processed for a variety of applications as described elsewhere herein. Those of ordinary skill in the art understand other sources available and may well be appropriate, such as those exemplified in the Table above.

### Fibroin Structure and Self-Assembly

The complete sequence of the Bombyx mori fibroin gene has been determined (C.-Z Zhou, F Confalonieri, N Medina, Y Zivanovic, C Esnault and T Yang et al., Fine organization of Bombyx mori fibroin heavy chain gene, Nucl. Acids Res. 28 (2000), pp. 2413-2419). The fibroin coding sequence presents a spectacular organization, with a highly repetitive and G-rich (−45%) core flanked by non-repetitive 5′ and 3′ ends. This repetitive core is composed of alternate arrays of 12 repetitive and 11 amorphous domains. The sequences of the amorphous domains are evolutionarily conserved and the repetitive domains differ from each other in length by a variety of tandem repeats of subdomains of ˜208 bp.

The silkworm fibroin protein consists of layers of antiparallel beta sheets whose primary structure mainly consists of the recurrent amino acid sequence (Gly-Ser-Gly-Ala-Gly-Ala)n (SEQ ID NO: 21). The beta-sheet configuration of fibroin is largely responsible for the tensile strength of the material due to hydrogen bonds formed in these regions. In addition to being stronger than Kevlar, fibroin is known to be highly elastic. Historically, these attributes have made it a material with applications in several areas, including textile manufacture.

Fibroin is known to arrange itself in three structures at the macromolecular level, termed silk I, silk II, and silk III, the first two being the primary structures observed in nature. The silk II structure generally refers to the beta-sheet conformation of fibroin. Silk I, which is the other main crystal structure of silk fibroin, is a hydrated structure and is considered to be a necessary intermediate for the preorganization or prealignment of silk fibroin molecules. In the nature, silk I structure is transformed into silk II structure after spinning process. For example, silk I is the natural form of fibroin, as emitted from the Bombyx mori silk glands. Silk II refers to the arrangement of fibroin molecules in spun silk, which has greater strength and is often used commercially in various applications. As noted above, the amino-acid sequence of the β-sheet forming crystalline region of fibroin is dominated by the hydrophobic sequence. Silk fibre formation involves shear and elongational stress acting on the fibroin solution (up to 30% wt/vol.) in the gland, causing fibroin in solution to crystallize. The process involves a lyotropic liquid crystal phase, which is transformed from a gel to a sol state during spinning—that is, a liquid crystal spinning process 1. Elongational flow orients the fibroin chains, and the liquid is converted into filaments.

Silk III is a newly discovered structure of fibroin (Valluzzi, Regina; Gido, Samuel P.; Muller, Wayne; Kaplan, David L. (1999). “Orientation of silk III at the air-water interface”. International Journal of Biological Macromolecules 24: 237-242). Silk III is formed principally in solutions of fibroin at an interface (i.e. air-water interface, water-oil interface, etc.).

Silk can assemble, and in fact can self-assemble, into crystalline structures. Silk fibroin can be fabricated into desired shapes and conformations, such as silk hydrogels (WO2005/012606; PCT/US08/65076), ultrathin films (WO2007/016524), thick films, conformal coatings (WO2005/000483; WO2005/123114), foams (WO 2005/012606), electrospun mats (WO 2004/000915), microspheres (PCT/US2007/020789), 3D porous matrices (WO2004/062697), solid blocks (WO2003/056297), microfluidic devices (PCT/US07/83646; PCT/US07/83634), electro-optical devices (PCT/US07/83639), and fibers with diameters ranging from the nanoscale (WO2004/000915) to several centimeters (U.S. Pat. No. 6,902,932). The above mentioned applications and patents are incorporated herein by reference in their entirety. For example, silk fibroin can be processed into thin, mechanically robust films with excellent surface quality and optical transparency, which provides an ideal substrate acting as a mechanical support for high-technology materials, such as thin metal layers and contacts, semiconductor films, dielectic powders, nanoparticles, and the like.

Unique physiochemical properties of silk allows its use in a variety of applications. For example, silk is stable, flexible, durable and biocompatible. Biocompatibility broadly refers to silk's safe and non-toxic nature, including being biodegradable, edible, implantable and non-antigenic (e.g., does not cause irritation or induce immune response). Furthermore, useful silk materials can be prepared through processes that can be carried out at room temperature and are water-based.

### Active Agents

In some embodiments, one or more active agents can be combined in silk fibroin solution for further processing into silk matrix, or can be otherwise introduced into a silk matrix or composition. The variety of active agents that can be used in conjunction with the silk matrix of the invention is vast. For example, the active agent may be a therapeutic agent or biological material, such as cells, proteins, peptides, nucleic acid analogues, nucleotides, oligonucleotides, nucleic acids (DNA, RNA, siRNA), peptide nucleic acids, aptamers, antibodies or fragments or portions thereof, hormones, hormone antagonists, growth factors or recombinant growth factors and fragments and variants thereof, cytokines, enzymes, antibiotics or antimicrobial compounds, anti-inflammation agent, antifungals, antivirals, toxins, prodrugs, chemotherapeutic agents, small molecules, drugs (e.g., drugs, dyes, amino acids, vitamins, antioxidants) and combinations thereof.

Exemplary antibiotics suitable for inclusion in the silk matrix of the invention include, but are not limited to, aminoglycosides (e.g., neomycin), ansamycins, carbacephem, carbapenems, cephalosporins (e.g., cefazolin, cefaclor, cefditoren, cefditoren, ceftobiprole), glycopeptides (e.g., vancomycin), macrolides (e.g., erythromycin, azithromycin), monobactams, penicillins (e.g., amoxicillin, ampicillin, cloxacillin, dicloxacillin, flucloxacillin), polypeptides (e.g., bacitracin, polymyxin B), quinolones (e.g., ciprofloxacin, enoxacin, gatifloxacin, ofloxacin, etc.), sulfonamides (e.g., sulfasalazine, trimethoprim, trimethoprim-sulfamethoxazole (co-trimoxazole)), tetracyclines (e.g., doxycyline, minocycline, tetracycline, etc.), chloramphenicol, lincomycin, clindamycin, ethambutol, mupirocin, metronidazole, pyrazinamide, thiamphenicol, rifampicin, thiamphenicl, dapsone, clofazimine, quinupristin, metronidazole, linezolid, isoniazid, fosfomycin, and fusidic acid.

Exemplary cells suitable for use herein include, but are not limited to, progenitor cells or stem cells, smooth muscle cells, skeletal muscle cells, cardiac muscle cells, epithelial cells, endothelial cells, urothelial cells, fibroblasts, myoblasts, chondrocytes, chondroblasts, osteoblasts, osteoclasts, keratinocytes, hepatocytes, bile duct cells, pancreatic islet cells, thyroid, parathyroid, adrenal, hypothalamic, pituitary, ovarian, testicular, salivary gland cells, adipocytes, and precursor cells.

Exemplary antibodies include, but are not limited to, abciximab, adalimumab, alemtuzumab, basiliximab, bevacizumab, cetuximab, certolizumab pegol, daclizumab, eculizumab, efalizumab, gemtuzumab, ibritumomab tiuxetan, infliximab, muromonab-CD3, natalizumab, ofatumumab omalizumab, palivizumab, panitumumab, ranibizumab, rituximab, tositumomab, trastuzumab, altumomab pentetate, arcitumomab, atlizumab, bectumomab, belimumab, besilesomab, biciromab, canakinumab, capromab pendetide, catumaxomab, denosumab, edrecolomab, efungumab, ertumaxomab, etaracizumab, fanolesomab, fontolizumab, gemtuzumab ozogamicin, golimumab, igovomab, imciromab, labetuzumab, mepolizumab, motavizumab, nimotuzumab, nofetumomab merpentan, oregovomab, pemtumomab, pertuzumab, rovelizumab, ruplizumab, sulesomab, tacatuzumab tetraxetan, tefibazumab, tocilizumab, ustekinumab, visilizumab, votumumab, zalutumumab, and zanolimumab.

In some embodiments, silk electronic components of the present invention further comprises a polypeptide (e.g., protein), including but are not limited to: one or more antigens, cytokines, hormones, chemokines, enzymes, and any combination thereof.

Exemplary enzymes suitable for use herein include, but are not limited to, peroxidase, lipase, amylose, organophosphate dehydrogenase, ligases, restriction endonucleases, ribonucleases, DNA polymerases, glucose oxidase, laccase, and the like.

Additional or alternative active agents suitable for use herein include cell growth media, such as Dulbecco's Modified Eagle Medium, fetal bovine serum, non-essential amino acids and antibiotics; growth and morphogenic factors such as fibroblast growth factor, transforming growth factors, vascular endothelial growth factor, epidermal growth factor, platelet derived growth factor, insulin-like growth factors), bone morphogenetic growth factors, bone morphogenetic-like proteins, transforming growth factors, nerve growth factors, and related proteins (growth factors are known in the art, see, e.g., Rosen & Thies, CELLULAR & MOLECULAR BASIS BONE FORMATION & REPAIR (R. G. Landes Co., Austin, Tex., 1995)); anti-angiogenic proteins such as endostatin, and other naturally derived or genetically engineered proteins; polysaccharides, glycoproteins, or lipoproteins; anti-infectives such as antibiotics and antiviral agents, chemotherapeutic agents (i.e., anticancer agents), anti-rejection agents, analgesics and analgesic combinations, anti-inflammatory agents, and steroids.

In some embodiments, an active agent may also comprise a cell, such as a bacterium, fungus, a plant or animal cell, and a virus.

In some embodiments, an active agent may include or be selected from neurotransmitters, hormones, intracellular signal transduction agents, pharmaceutically active agents, toxins (such as chemical toxins, biological toxins, e.g.) and other toxic agents, agricultural chemicals, microbes, and animal cells, such as neurons, liver cells, immune cells and stem cells.

The active agents may also include therapeutic compounds, such as pharmacological materials, vitamins, sedatives, hypnotics, prostaglandins and radiopharmaceuticals.

An active agent for use in accordance with the present invention may be an optically or electrically active agent, including but not limited to, chromophores; light emitting organic compounds such as luciferin, carotenes; light emitting inorganic compounds, such as chemical dyes; light harvesting compounds such as chlorophyll, bacteriorhodopsin, protorhodopsin, and porphyrins; light capturing complexes such as phycobiliproteins; and related electronically active compounds; and combinations thereof.

In some embodiments, active agents to be used in accordance with the present invention are pharmaceutical agents (e.g., drugs) that are inclined to cause adverse or unwanted side effects in patients when administered systemically at a dose known to be therapeutically effective. In some embodiments, drugs that cause adverse side effects in patients when administered systemically are anti-cancer agents, including chemotherapeutics.

Generally, chemotherapy constitutes an important part of cancer treatment for many cancer patients. Chemotherapy can be used to destroy cancer cells, stop cancer cells from spreading (metastasis), and/or slow the growth of cancer cells. Chemotherapy can be given alone (e.g., monotherapy) or in conjunction with other treatments (e.g., combination therapy). It can have synergistic effects, such that it helps other treatments work better. For example, chemotherapy may be given before or after surgery or radiation therapy. In some cases, chemotherapy may be given before a peripheral blood stem cell transplant.

For example, patients who undergo a resection surgery to remove a solid tumor may receive a chemotherapy as part of a post-operative treatment regimen, typically referred to as adjuvant chemotherapy. Adjuvant chemotherapy has been shown to provide a substantial benefit for cancer patients. Studies have shown improved survival in patients with a variety of cancers, such as early stage lung cancer and colorectal cancer, who receive chemotherapeutics following surgical treatment.

### Side Effects

While chemotherapy can be an effective cancer treatment, it has also been associated with a number of unwanted, adverse side effects. Common side effects that accompany cancer chemotherapy include, but are not limited to: anemia, appetite changes, bleeding problems, constipation, diarrhea, fatigue, hair loss (alopecia), infection, memory changes, mouth and throat changes, nausea and vomiting, nerve changes, pain, sexual and fertility changes in men, sexual and fertility changes in women, skin and/or nail changes, swelling (fluid retention), and urination changes. Many of these side effects of chemotherapeutics are particularly prevalent in chemotherapeutics that are administered systemically (e.g., intravenous administration, oral administration, etc.), as opposed to locally.

Most chemotherapeutics in use today are designed for systemic administration. A large proportion of patients who receive chemotherapy experiences moderate to severe side effects at a dose effective to treat cancer. Needless to say, such adverse effects of cancer treatment have a significant effect on the quality of life for patients undergoing treatment.

The present invention encompasses the recognition that certain drugs may be delivered locally, rather than systemically, to give a beneficial effect. According to the invention described herein, silk-based delivery devices (e.g., silk fibroin delivery matrices) can be fashioned to accommodate local delivery of therapeutics, including chemotherapeutics. In this way, the present invention provides a way to reduce adverse side effects of a therapeutic agent caused by systemic administration, while providing comparable effectiveness at the same or similar dose. In some embodiments, it is possible to achieve equivalent therapeutic effects of a drug using a lower dosage that is administered locally to a site of interest. In some embodiments, such administration has an effect on a target tissue, without significantly affecting other tissues or organs of the body.

### Local Delivery

In some embodiments, a subject who may benefit from local delivery of a therapeutic agent has a localized lesion. A localized lesion as used herein may refer to any target part(s) of a subject's body (regions, tissues, organs, cells, etc.) that is not systemic. For example, in some embodiments of the invention, localized lesions may comprise diseased or abnormal tissues, injured tissues, surgical, incision and/or injection sites, including those associated with tissue repair and reconstruction, cell and tissue harvesting, as well as cosmetic and plastic surgeries. In some embodiments, localized lesions may refer to cancerous tissues, such as solid tumors. In some embodiments, localized lesions may refer to the site of surgery, such as the resection cavity, which results from a surgical procedure. In some embodiments, resection cavities are formed as a result of the removal of an affected tissue or organ, e.g., tumors, injured tissues, etc. According to the invention, silk fibroin delivery matrices described herein may function as a useful platform for delivering a therapeutic agent to the target area of interest in a subject's body with minimal systemic effects.

Typically, silk fibroin delivery matrices described herein are implanted within the body of a subject. In some embodiments, for example, a silk fibroin delivery matrix carrying at least one agent (e.g., drugs) can be positioned just below the surface of the subject's skin, for example about 0-4 mm deep, e.g., about 1 mm, about 2 mm, about 3 mm, about 4 mm below the surface of the skin. In some embodiments, a silk fibroin delivery matrix carrying at least one agent (e.g., drugs) can be positioned deeper into tissues, e.g., about 4-60 mm below the surface of the subject's skin, e.g., about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 25 mm, about 30 mm, about 40 mm about 50 mm about 60 mm, below the surface of the subject's skin.

Where real time monitoring of sustained drug delivery is desirable, typically such a device is positioned no more than 15 mm below the surface of the skin, so as to enable detection/measurement of optical signals from an external detector. However, in situations where it is not crucial to monitor drug delivery following implantation of a silk fibroin delivery matrix, the positioning of such a device with respect to the depth as measured from the surface of a skin may not be critical.

Implantation may be accomplished by or during surgery, by injection, etc.

In some embodiments, subjects having a localized lesion are prone to develop an infection at the site of the lesion, which may include, for example, surgical or incision site, or an injured tissue that requires repair or reconstruction. According to the invention, silk fibroin delivery matrices described herein are useful for preventing and/or treating infection by locally delivering an anti-infection agents such as antibiotics to the site where it is needed. Some embodiments include the use of provided silk delivery matrices as part of surgical procedures, such that such a device can be positioned or implanted at the time of surgery or incision as part of a preventive measure to fight infection. There is no need for removing the device later on, since the device is designed to fully resorbed into tissues.

Such applications may be particularly desirable for surgical resections, such as removal of a solid tumor. It is contemplated that following the removal of a tumor from a patient, a silk fibroin delivery matrix described herein can be placed within the resection cavity or near the site of surgical incision. The fibroin delivery matrix will degrade over time, while locally releasing a therapeutically effective amount of a drug that is incorporated into the delivery matrix. In some embodiments, a drug or drugs to be delivered by such a method may include a chemotherapeutic agent, antibiotics, etc. In this way, subjects subsequently receive a sustained dose of desired therapeutics at the target site. Such methods can help prevent infections, and/or promote cancer cell killing, so as to reduce the probability of metastasis. Moreover, where desired, such a delivery device may alternatively or additionally comprise biologically active agent(s) to promote tissue repair, wound healing, etc.

In some embodiments of the invention, silk fibroin delivery matrices may be used to deliver an agent locally but for systemic effects. For example, a silk fibroin delivery matrix comprising a drug can be strategically positioned in a patient, e.g., near a major blood vessel, such that the drug incorporated in the delivery matrix is slowly released into the body of the patient then enters the blood stream at a rate relative to the rate of degradation of the silk matrix itself.

### Sustained Release

Silk fibroin delivery matrices embraced by the present invention are also useful in providing a means for a long-term, slow, and/or sustained release of an agent into a body. In some embodiments, such long-term, slow, and/or sustained release of an agent may be designed for a systemic effect and/or a local effect. Such application may be particularly suitable in situations in which an agent to be administered gives favorable therapeutic effects when the bioavailability of the agent (e.g., a drug) is maintained at certain levels in the body for a prolonged period of time. In some embodiments, a drug to be administered for a sustained release has a relatively short half-life in vivo (e.g., under physiological conditions).

In some embodiments, subjects who may benefit from a sustained release of an agent is susceptible to developing or has developed a chronic disease, disorder or condition. In some embodiments, subjects who may benefit from a sustained release of an agent has a genetic disorder. In some embodiments, subjects who may benefit from a sustained release of an agent is in need of a prolonged therapy, including but are not limited to, enzyme replacement therapy and hormone replacement therapy. In some embodiments, subjects who may benefit from a sustained release of an agent has an immune disorder, including an autoimmune disorder.

### Optical Features

As already alluded to, silk-based materials can be fabricated to provide certain favorable structural and optical properties. As such, multifunctional devices described herein are in some embodiments implantable optical devices. In some embodiments, such devices include optical reflectors comprised of biocompatible and bioresorbable silk fibroin materials, thus providing an implantable component/device of optical utility, and capable of being incorporated in vivo. For example, provided silk retroreflectors may utilize millimeter size microprism arrays to rotate the image plane of imaged cortical layers, thus enhancing the amount of photons that are detectable in the reflected direction when inserted in a sample to be analyzed, and ultimately increasing the contrast ratio in multiphoton microscopy. Developing such material platforms addresses the need in medical imaging and diagnosis field for materials that are suited for optical and photonic component fabrication and at the same time can be introduced inside the human body without the need for retrieval. Detailed structural features of silk optical components and their optical features are described in, for example, WO 2011/046652, the entire contents of which are incorporated herein by reference.

Suitable reflective elements useful for the present invention may be a single reflective element or reflective elements in a ID, 2D or 3D array. Such reflective elements may be mirrors and retroreflectors with various shapes and geometries, including but not limited to flat mirrors, diamond-cut reflectors, retroreflectors with geometries such as a corner-cube, hemispherical geometry, “cat's-eye” geometry or the mirror-backed lens (see, e.g., Lundvall et al., 11 Optics Express, 2459 (2003)), retro-reflecting cavities containing plurality of orthogonal intersecting planes, such as the corners of square, rectangular, or cubical cavities.

The term “retroreflective” as used herein refers to the attribute of reflecting an obliquely incident light ray in a direction antiparallel to its incident direction, or nearly so, such that it returns to the light source or the immediate vicinity thereof. Retroreflectors can, over a broad angle, return light toward its source. Hence they are highly detectable by using simple illumination and detection with or without spectral filters. Retroreflectors may be used in a wide range of applications from retroreflective paints to enhance reflective brightness on signs or markers for macroscale retroreflectors, to biological recognition elements in medical imaging, bioassays or biosensors for microscale retroreflectors. Thus, favorable optical properties in an implantable device of the present disclosure include reflective property.

Challenge in realizing the operativity of such a multifunctional device includes achieving sufficient signal-to-noise differential in an in vivo context. That is, an external sensor (e.g., a detector) must be able to detect and measure optical signals (e.g., reflection) from an implanted silk fibroin device often through layers of tissues. Additional challenge relates to maintaining independent functionality of different modalities of a device, while structurally incorporating theses modalities.

The work described in the present application demonstrates the confluence of optical form and biomedical function in one system, by manufacturing implantable, multifunctional, bioresorbable micro-optical devices. The results demonstrate a next generation concept that has reached reality, opening the door to new medical device designs that can impact health care in many modes.

In the context of the present disclosure, a device is said to be multifunctional in that such a device represents a platform such that: (1) silk can be formed into an optical element that sits within tissue; (2) through the properties of the silk users can stabilize and preserve the efficiency of agent(s) (such as chemotherapeutic drugs) which otherwise degrade—not only from temperature, but photodegrades; (3) then as the drug is delivered the reflectivity changes allowing one to monitor how much drug is released (see Exemplifications). The coexistence of these properties in one single device does not exist in prior art. Importantly, as one of ordinary skill in the art will recognize, the fact that optical performance is comparable in the doped and undoped case, as demonstrated here, is not a given. While many biopolymers can be doped, they do not function as an optical element or do not stabilize the labile drugs or localized therapeutic absorbers to a predesigned geometry.

### Other Functionalization

As stated above, functionalization of an optical device, such as a silk fibroin matrix, involves incorporation of an independently functional agent to the device, in such a way that the functionality is correlated with the structure of the device itself. In some embodiments, a functional agent to be incorporated into a silk fibroin matrix comprises a plurality of particles, including but are not limited to nanoparticles. In some embodiments, these may comprise plasmonic nanoparticles, metal-based nanoparticles, etc., with independent functionality. In some embodiments, useful plasmonic nanoparticles can form a silk-based photothermal element. As a basis for generating heat useful for the present invention, certain nano-scale heating elements, such as plasmonic nanoparticles (e.g., GNP and gold nanoshells (GNS)), may be used. Thus in the context of the multifunctional optical devices of the present disclosure, incorporation of nanoparticles into a silk matrix does not interfere with the optical function of the silk structure, such as microprisms. At the same time, the resulting nanoparticles incorporated into the silk matrix still maintains function, such as thermogenesis.

According to the invention, it is further contemplated that devices that comprise a heating element provide a wide range of biomedical and clinical applications, such as thermal therapy. In particular, light-activated heating elements are of great interest for a number of applications, including photothermal therapy, in which electromagnetic radiation is employed to treat various medical conditions. Because silk can be doped with a variety of materials, the invention described herein can be used to design a silk-based lattice or mould (e.g., silk film) comprising a light-activated heating element when combined with plasmonic nanoparticles. Such combination can produce photothermal device of superior features, as compared to those previously described in the art. For example, to support this novelty, silk fibroin films have been reformed into ribbons with AuNP to illustrate the use of local heating as a heating/cauterizing/therapeutic suture. In some embodiments, photothermal elements incorporated in a silk fibroin matrices can provide temperature of about 40° C., 41° C., 42° C., 43° C., 44° C., 45° C., 46° C., 47° C., 48° C., 49° C., 50° C., 51° C., 52° C., 53° C., 54° C., 55° C. or higher.

In some embodiments of the present invention, the photothermal element provides for a system that can modulate in vivo delivery of an agent. The system includes a plurality of plasmonic nanoparticles, capable of converting incident radiation into heat energy when the nanoparticles are irradiated with electromagnetic radiation, contained in a silk fibroin matrix that can further comprise at least one active agent distributed therein. By way of example, when the temperature of the silk fibroin matrix or portion thereof is at a first temperature (e.g., 37° C.), the active agent is retained within the silk fibroin matrix. When the silk fibroin matrix or a portion thereof is raised to a second, higher temperature (e.g., ˜40° C.-45° C.), i.e., heat generated by plasmonic particles exposed to electromagnetic radiation, at least a portion of the active agent can be released from the silk fibroin matrix into the body. Optionally, embodiments of the invention can include a biosensor system, e.g., for providing information about in vivo status to assist in making treatment decisions. An advantage of the system is the ability to locally change the temperature of a thermally-responsive IMD by exposure to light targeted for absorption and conversion to heat by plasmonic nanoparticles (including, e.g., metal nanoshells). This allows implantation of a drug delivery device with multiple dosages, and provides for an external control over the dosage profiles by regulating exposure of the drug delivery device to an appropriate light source.

Another aspect of the invention relates to a method of photothermally modulating in vivo delivery of an active agent. The method includes implanting into the body of a subject in need of treatment, a composition or a device containing one or more plasmonic nanoparticles and at least one active agent in a silk fibroin matrix. The active agent can be substantially retained by the silk fibroin matrix when the temperature of the composition is at about normal body temperature of the subject. At least a portion of the active agent can be substantially released from the silk fibroin matrix into the body of the subject when the temperature of the composition, or a portion thereof, is raised. The method includes applying electromagnetic radiation, such as near-infrared radiation, to the implanted composition or device from outside the body. The electromagnetic radiation can be applied through an optical grid. The amount and duration of electromagnetic radiation can be applied until it is sufficient to raise the temperature of the plasmonic nanoparticles such that the silk fibroin matrix, or a portion thereof, can cause release of the agent to commence. Alternatively, application of the electromagnetic radiation can be continued until a desired amount of the active agent has been released from the implant into the body. After the desired amount of the agent has been delivered, the composition can be allowed to return to normal body temperature, whereupon drug delivery is reduced or ceased, as desired. In some embodiments, the application of electromagnetic radiation can be repeated at a later time, if multiple dosing is desired. In some embodiments, the treatment method can further comprise applying ultrasound, magnetic fields, electric fields, or any combinations thereof, to the implanted composition or device from outside the body. The silk fibroin matrix is biocompatible and biodegradable, and does not require subsequent removal. The implantation can be subcutaneous or parenteral.

### Silk-Doxorubicin Delivery Matrix

In some embodiments, the multifunctional device described herein is used as a platform for sustained delivery of at least one agent (e.g., drugs, biological agents, enzymes, hormones, etc.). In some embodiments, such delivery may comprise systemic administration. In some embodiments, such delivery may comprise local administration.

A silk fibroin delivery device described herein can be placed in a subject at a desired location(s); subsequently, in vivo release can be monitored over time by measuring changes in optical features of the silk delivery matrix.

As described, in exemplary embodiments of the invention, a silk-based delivery device comprises a silk fibroin matrix such as silk film, doped with a therapeutic agent, such as chemotherapeutics, to be locally delivered in a subject in need thereof. In some embodiments, a silk fibroin matrix may be further fabricated to comprise a reflective unit, typically an array of microprisms. Non-limiting examples of chemotherapeutic agents useful in the application of the present disclosure include doxorubicin.

Doxorubicin is among the most effective chemotherapeutics used for the treatment of cancers including breast, ovarian, sarcomas, pediatric solid tumors, Hodgkin's disease, multiple myeloma, and non-Hodgkin's lymphomas. Doxorubicin acts primarily by forming a stable ternary complex with DNA and topoisomerase II. Despite its broad specificity against many cancers, the clinical use of doxorubicin has been severely limited due to its side effects, particularly its severe cardiac toxicity. Efforts to improve the safety and efficacy of doxorubicin have included encapsulation in polymeric micelles, conjugation to synthetic polymers and the addition of targeting antibodies. Though these approaches all reduce toxicity, none of them demonstrable improve efficacy (Cai et al., 2010).

A delivery system for doxorubicin is therefore needed that achieves high local concentrations of the drug while also limiting exposure of vulnerable cardiomyocyes to both maximize safety and efficacy. Controlled, sustained release drug carriers have the potential to meet the need for local delivery, with the added benefits of reduced frequency of administration, improved patient convenience and compliance and drug levels that are continuously maintained in a therapeutically desirable range without peaks and valleys (Langer, 1980).

As already stated above, silk fibroin (a biologically derived protein polymer isolated from the cocoons of the domestic silkworm (Bombyx mori)) has been investigated for implantable and injectable sustained drug delivery applications due to its unique properties. Silk possesses excellent biocompatibility (Leal-Egana and Scheibel, 2010; Tang et al., 2009; Meinel et al., 2005; Seo et al., 2009; Panilaitis et al., 2003), robust mechanical strength (Altman et al., 2003) and has been shown to support cell growth, proliferation and differentiation (Acharya et al., 2008; Wang et al., 2006). Silk degrades to non-toxic products in vivo and the degradation time course of silk implants can be controlled from weeks to years via regulation of beta sheet content (crystallinity) during processing (Horan et al., 2005; Wang et al., 2008-1; Numata and Kaplan, 2010). Unlike synthetic polymeric drug carrier systems, which require harsh manufacturing conditions that can degrade incorporated therapeutics (such as shear, heat, organic solvents or extreme pH), silk can be processed entirely in aqueous systems using mild, ambient conditions of temperature and pressure (Vepari and Kaplan, 2007; Lawrence et al., 2008). Stable, physical crosslinking of silk can be achieved during the crystallization process to form beta sheets, negating any need for chemical crosslinking and thereby avoiding potentially toxic chemicals. Further, silk has been found to exert a significant stabilizing effect on encapsulated enzymes and antibodies, even at elevated storage temperatures (Lu et al., 2009; Lu et al., 2010-; Guziewicz et al., 2011).

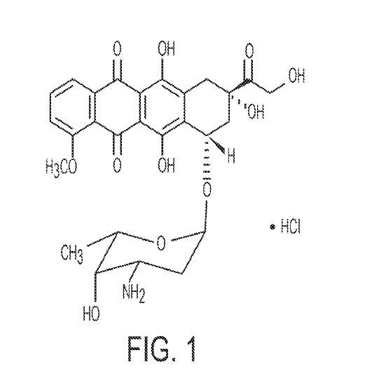

In addition to having properties which make silk well suited to drug delivery in general, silk is uniquely suited to deliver doxorubicin, as we have observed that doxorubicin interacts strongly with the silk. This results in sustained diffusion-driven release of the freely diffusible doxorubicin loaded in a silk carrier, despite the relatively small size of the doxorubicin (mol. wt.=579.98, see FIG. 1), even in a highly diffusive matrix like a hydrogel. The interaction between the silk and the doxorubicin also results in a large fraction (typically more than 50%) of the total doxorubicin dose is bound to the carrier. In some embodiments, therefore, the carrier may be proteolytically degraded to enhance release the bound doxorubicin. This provide an opportunity to design protease-triggered drug release systems. Several disease states (particularly cancer) have been shown to increase local proteolytic degradation (Law and Tung, 2009). Degradation-mediated drug release from silk biomaterials can be used to design systems that provide drug locally proportionately to disease progression: aggressive disease/increased proteolysis would degrade the silk carrier more rapidly, releasing larger doses of drug than in healthy tissue.

The following exemplification represents non-limiting embodiments of the present invention and is not to be construed in any way to be limiting.

### EXEMPLIFICATION

### Example 1

### Doxorubicin Silk Films

To characterize doxorubicin release and retention, two types of silk drug carriers, silk films (implantable delivery) and silk hydrogels (injectable delivery) were investigated both in vitro and in vivo. Additionally, stability of doxorubicin encapsulated in silk films compared with storage in solution, and release behavior versus degradation, were also examined.

The fabrication methods used for preparing silk fibroin materials are as follows: Bombyx mori fibroin solution was prepared as described previously (Biomaterials 2005 26 2775). Briefly, cocoons were cut into ˜25 mm² pieces, boiled for approximately 30 minutes in an aqueous solution of 25 mM NaCO₃ and then rinsed in ddH₂O to remove sericin proteins. Extracted silk fibroin was air-dried and subsequently dissolved it in 9.3M LiBr solution at 60° C. for 4 h, yielding a 20 wt % solution. This solution was dialysed against ddH₂O (molecular weight cut off 3,500) for ˜48 h to remove the LiBr salt. The resulting aqueous silk fibroin solution was centrifuged twice at 9.700 g for 20 min to remove the small amounts of silk aggregate that formed during processing. The final concentration of the aqueous silk solution was ˜7.5 wt % as determined by weighing the remaining solid after drying. Silk films were prepared by casting 4 ml of 4 wt % silk fibroin onto 25 cm² polydimethylsiloxane templates and drying at 25° C. and 60% relative humidity. Films were generated with a graded amount of cross linking (e.g., β-sheets) by either autoclaving or water annealing them as detailed elsewhere (Biomacromolecules 2011 12 1686). Films were loaded with doxorubicin by adding the drug to the silk fibroin solution prior to casting. Alternatively, cross-linked silk films were immersed in an aqueous doxorubicin solution and the amount of dug loading was monitored at 508 nm. The latter approach is well suited for cross linking conditions that are deleterious to the drug.

Silk films for degradation and stability studies were prepared by casting of 6% (w/v) silk solution containing 1.0 mg/mL doxorubicin on a patterned mold. After drying at ambient conditions for 48 hours the samples were removed and vapor annealed for 24 hours as previously described (Jin et al., 2005).

### Example 2

### In Vitro Drug Release

### Drug Release Profile was Studied In Vitro

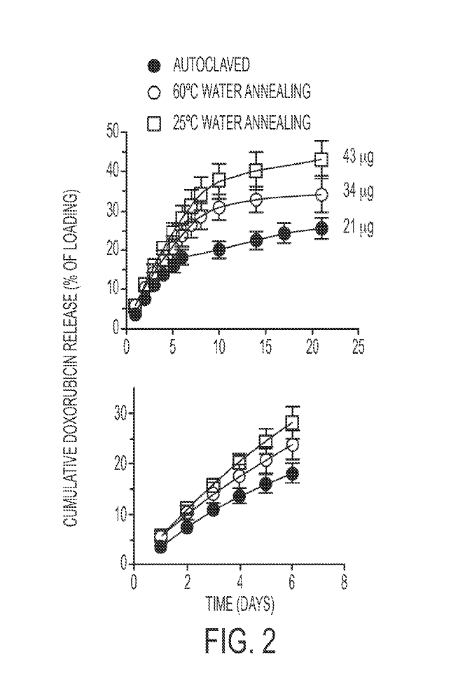

First, the release of doxorubicin from silk films was monitored by incubating them in PBS at 25° C. and measuring the amount of drug release by determining doxorubicin-associated fluorescence (excitation 480 nm and emission at 590 nm) (FIG. 2). Increased beta-sheet content in the silk films (autoclaved>60° C. water annealing>25° C. water annealing) decreases release rate and increases total cumulative recovery. Zero-order, constant DOX release is observed for the first 6 days of release (lower graph).

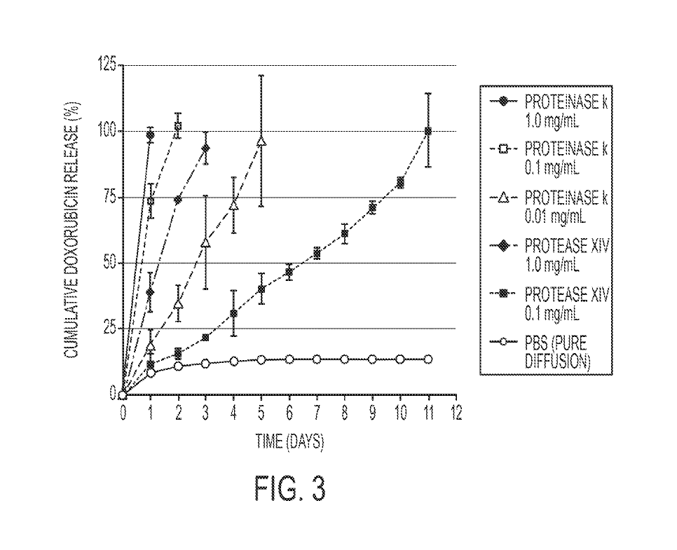

Next, effects of proteolytic enzyme in the process were evaluated. Doxorubicin release from silk films in PBS (no enzyme as control) and buffer containing varied concentrations of two commonly studied silk-degrading proteinases (proteinase k and protease type XIV) is shown in FIG. 3. Values of t₅₀ (the time at which 50% of the total drug load is released) are reported in Table 3 provided below.

TABLE 3
Time at which 50% of the total drug load was released (t₅₀) for various
proteolytic buffer compositions and concentrations

| | Concentration | t₅₀ |
|---|---|---|
| | (mg/mL) | (days) |
| Proteinase k | 1.0 | 0.51 |
| (approx. 30 units per mg) | 0.1 | 0.68 |
| | 0.01 | 2.66 |
| Protease type XIV | 1.0 | 1.31 |
| (approx. 3.5 units per mg) | 0.1 | 6.49 |

Increased proteolytic degradation (proteinase k degrades silk more aggressively than protease type XIV; increasing proteinase concentration increases rate of silk film degradation) decreases the time at which 50% of the total drug load is released (t₅₀). Films in PBS (no enzyme present, release is purely diffusional) release only 13.4% of the total drug load over 7 days, suggesting that while some doxorubicin is freely diffusible, complete drug release requires proteolytic degradation.

### Example 3

### Doxorubicin Stabilization in Silk Films

Doxorubicin is known to be highly susceptible to photodegradation (Wood et al., 1990) and increased degradation is observed with increased storage temperature (Law et al., 1991). However, encapsulation in liposomes has been shown to retard doxorubicin degradation (Bandak et al., 1999). Evidence indicated that silk encapsulation may reduce light- and/or temperature-induced degradation of antibiotics (unpublished result). Therefore, we hypothesized that silk could exert similar stabilizing effects on encapsulated doxorubicin. Doxorubicin in ultrapure water (two concentrations, 0.8 mg/mL and 7 mg/mL) and doxorubicin-loaded silk films were stored at −20° C. and 60° C. for 3 weeks. After 3 weeks, the silk films were degraded with 10 mg/mL proteinase k for 6 hours, and doxorubicin concentration for all samples was determined by comparing fluorescence to a standard curve of known concentrations (excitation=430, emission=550).

Results indicate that doxorubicin fluorescence decreases when stored in solution at all concentrations and temperatures. By comparison, the fluorescence of the doxorubicin stored in silk films does not significantly decrease at either at −20° C. or 60° C., despite the 80° C. increase in storage temperature, demonstrating that silk fibroin matrices such as silk films can stabilize an agent that is otherwise susceptible to degradation.

### Example 4

### Cell Culture and Cytotoxicity Assays

Breast cancer cell lines MDA-MB-231 and MCF-7 were obtained from ATTC (Manassas, Va., USA). All cell lines were maintained in a humidified atmosphere of 5% CO₂ at 37° C. and cultures were routinely subcultured every 2-3 days. MDA-MB-231 cells were grown in RPMI 1640 with 10% v/v FBS and MCF-7 cells in DMEM (4.5 g glucose, 110 mg sodium pyruvate) supplemented with 10% v/v FBS and 10 m/ml insulin. The in vitro toxicity of doxorubicin-loaded silk on these cells was examined by plating 2×10⁴ cells/cm² and allowing the cultures to recover for 24 h. Next, we added the doxorubicin silk substrates to the cultures and monitored cell viability. For endpoint studies we examined cell viability after a 72 h exposure time by using (3-(4,5-Dimethylthiazol-2-yl)-2,5-diphenyltetrazolium bromide (MTT at 5 mg/ml) as a substrate (J Drug Targeting 2006 14 375). We also examined the long term performance of the doxorubicin silk substrates by including 10% AlamaBlue to the culture medium for 4 h and determining cell viability according to the manufacturer's protocol.

### Example 5

### In Vivo Performance of Doxorubicin Silk Films

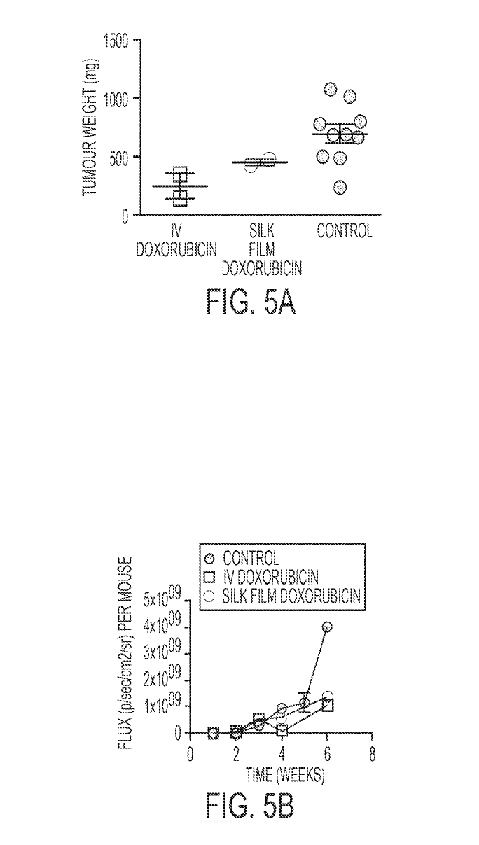

Female NOD/SCID mice aged 6 to 10 weeks were purchased from Charles River. In vivo studies were approved by Institutional Animal Care and Use Committee (IACUC) (Protocol M2010-101), and animals were maintained under the guidelines established by the NIH and Tufts University. We examined the therapeutic potential of doxorubicin-loaded silk films to slow tumour progression in vivo. For surgeries animals were anesthetised using isoflurane, shaved and the surgical area was cleaned. We injected 5×10⁴ luciferase expressing MDA-MB-231 cells bilaterally into the 4^{th} or 5^{th} mammary fat pad as detailed previously (Cancer Research 2010 70 10044). After 2 weeks of tumour induction, we subjected mice to a second round of surgery and implanted doxorubicin-loaded silk films at the primary tumour site. Tumour progression was monitored by injecting luciferin i.p. and detecting luminescence using Xenogen imaging. At the endpoint of the study, primary tumours were removed and weighed. Results of in vivo evaluation are shown in FIGS. 5A and 5B.

### Example 6

### Doxorubicin Silk Hydrogels

The fabrication methods used in the studies were as follows: Silk hydrogels were prepared using the sonication-induced gelation technique previously described (Wang et al., 2008). Briefly, bulk loaded gels were prepared by sonicating silk solution of the desired concentration and the desired degumming time using a Branson Digital Sonifier 450 at 15% amplitude for 60-90 seconds. Doxorubicin solution was mixed into the sonicated silk solution prior to the onset of gelation, aliquoted, then incubated at room temperature for 10-15 minutes to allow gelation and entrapping the doxorubicin. Silk hydrogel concentrations tested were 2% (w/v), 4% (w/v) or 8% (w/v); degumming times tested were either 20 minute or 45 minute and doxorubicin loadings tested were 1 mg/mL, 0.5 mg/mL, 0.25 mg/mL or 0.125 mg/mL (except for the 1 mg/mL, which was only loaded into 4% (w/v) and 8% (w/v) hydrogels as this loading was too high or silk concentration too low for gelation to occur).

### Example 7

### In Vitro Release Studies

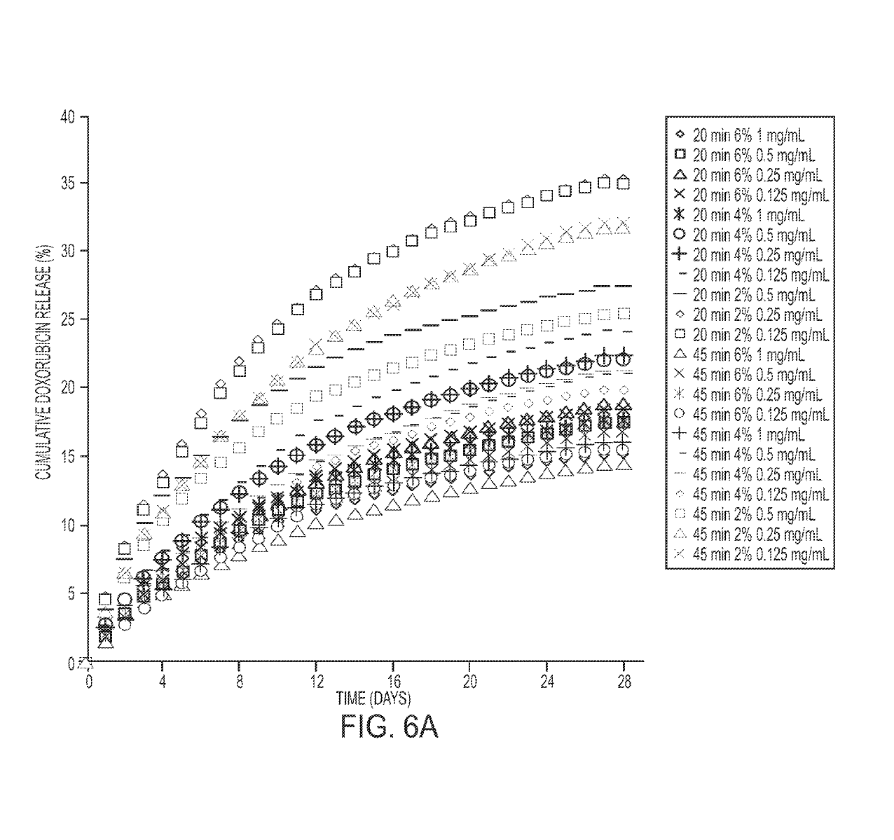

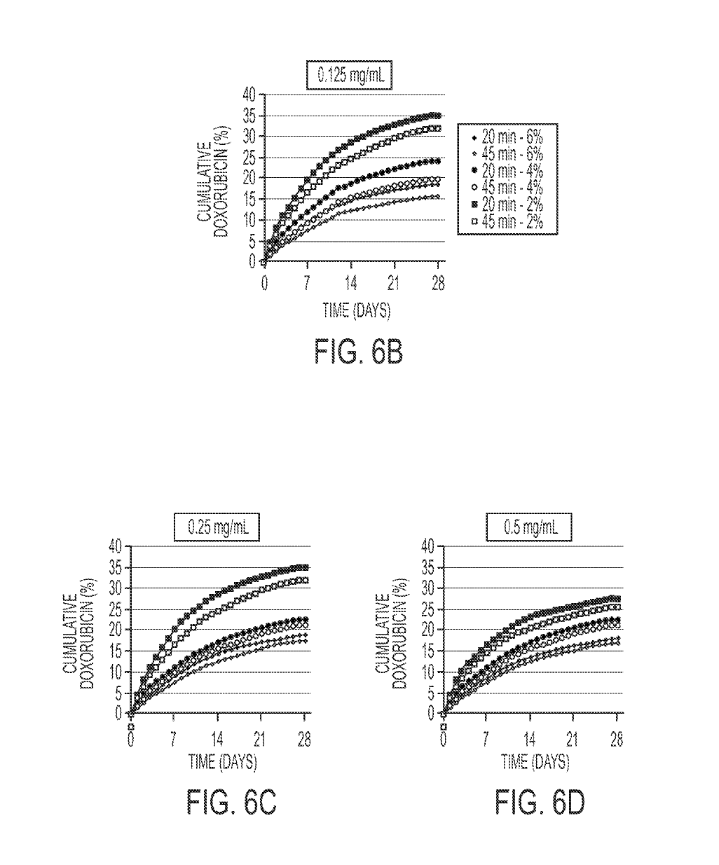

Release was determined by immersing drug loaded silk materials in 0.5 mL of Dulbecco's PBS at 37° C., removing and replacing the buffer every 24 hours, and measuring the amount of drug release by determining doxorubicin-associated fluorescence (excitation 480 nm and emission at 590 nm). Release for all hydrogel compositions/loadings tested are shown in FIG. 6A. The data shown in FIG. 6A are also represented in FIGS. 6B-6D, broken down by doxorubicin loading for ease of comparison.

Decreasing silk concentration increases release rate and total cumulative release (less silk in the same volume of hydrogel lowers retention). The longer degumming time (45 min compared with 20 min), the greater the reduction of drug release and cumulative release (longer degumming time reduces retention). The difference in doxorubicin loading from 0.25 mg/mL to 0.125 mg/mL does not substantially impact release, but with the increase to 0.5 mg/mL, release rate and cumulative from the 2% hydrogels (but not the 4% (w/v) or 6% (w/v)) decreased. This suggests that, apart from very high drug to silk ratios (0.5 mg DOX: 20 mg silk), loading does not significantly alter release behaviour.

### Example 8

### In Vitro Cell Culture and Cytotoxicity Studies

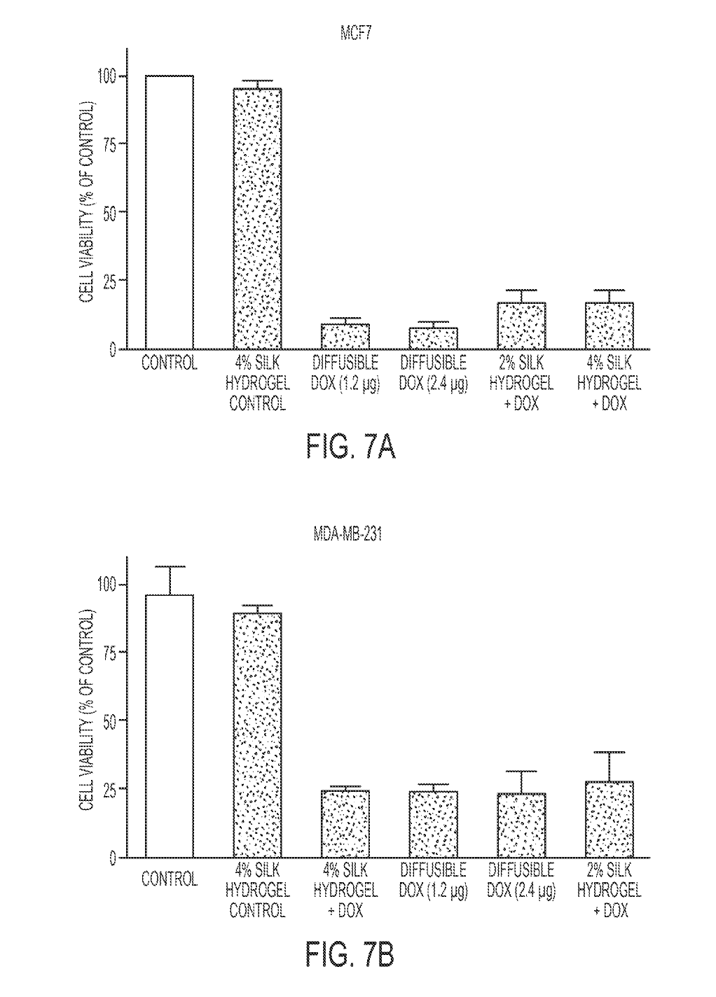

Cytotoxicity was evaluated as described for silk films, using transwell inserts filled with 100 μL of silk hydrogel (prepared as described above) loaded with a total dose of doxorubicin of 40 mg and 1×PBS. Loading was held constant (40 μg), but two silk hydrogel concentrations were tested (2% (w/v) and 4% (w/v)) to vary release behaviour. Cells were also exposed to soluble doxorubicin in concentrations approximating the dose released from the hydrogel (estimated based on in vitro release study). Cells were also exposed to empty silk hydrogel and media without doxorubicin as controls. Results are summarized in FIG. 7.

### Example 9

### Additional Drug Delivery Applications

Though more studies are needed to understand the mechanisms and control points for drug binding, several compounds besides doxorubicin have been identified as having a strong interaction with silk or beta-sheet, for example the beta-sheet binding dyes Congo Red (DaSilva et al., 2010) and Alcian Blue (Lammel et al., 2010). While dyes have limited therapeutic relevance compared to doxorubicin, they suggest that degradation-triggered release from silk could be extended to other therapeutic needs.

For example, crystal violet is known to bind beta-sheet (Askansas et al., 1993) and stain silk (Szybala et al., 2009), and has antibacterial, antifungal, and anthelmintic properties (Docampo and Moreno, 1990). Lammel et al. reported incomplete diffusional release of crystal violet from silk microspheres, and demonstrate the potential to control recovery via manipulation of compound-silk interactions: 25-60% is released depending on processing pH, which in turn controls silk II content, which theoretically dictates the extent of crystal violet's binding to the silk (Lammel et al., 2010). We also observed incomplete recovery of another antibiotic, rifampicin, from silk films in PBS (approx. 50% after 14 days for uncoated films, 30-40% from rifampicin loaded film reservoirs coated with an additional silk film barrier) (Pritchard et al., 2011). Infected wounds have been shown to possess higher levels of proteolytic enzymes than healthy tissue (Tanihara et al., 1999; Suzuki et al., 1997). We contemplate that the loading of silk biomaterials with antibiotics which have strong interactions with the silk could produce materials which release more antibiotic in response to increased proteolytic degradation.

In addition, we contemplate the silk could be chemically decorated with binding peptides to delay release of small molecules which otherwise do not interact with the silk.

### Example 10

### Multifunctional Silk Fibroin Platform

As described herein, the concept of a multifunctional device with a direct structural and functional relationship was put to test for the first time and has been optimized to bring it to be operative.

Free-standing two-dimensional (2D) micro-prism arrays (MPA) prepared solely from purified/reconstituted silk protein serves as the optical platform in the present work. This system provides optical signal and contrast enhancement by retroreflecting forward scattered photons through layers of tissue, causes no adverse biological effects and is slowly degraded and integrated into native tissue in vivo. Optical signal and contrast enhancement allow for improved non-invasive imaging of tissue and hence diagnostics. Additionally, the utility of the silk MPAs is augmented by incorporating biochemical function to demonstrate multifunctional optical elements.

To demonstrate that the enhanced reflectivity of this device is not compromised by functionalizing the silk MPA, dopants have been included in the silk material, which in this work are either gold nanoparticles (Au-NPs) or the chemotherapeutic drug doxorubicin. Furthermore, the resulting functional silk microreflector device doped with doxorubicin not only shows enhanced reflectivity offered by the optical device but allows for storage, controlled delivery and imaging of therapeutics. The optical performance of the reflector provides important transduction and monitoring mechanisms, since changes in reflectivity of the dissolving device can be correlated to the amount of drug eluted.

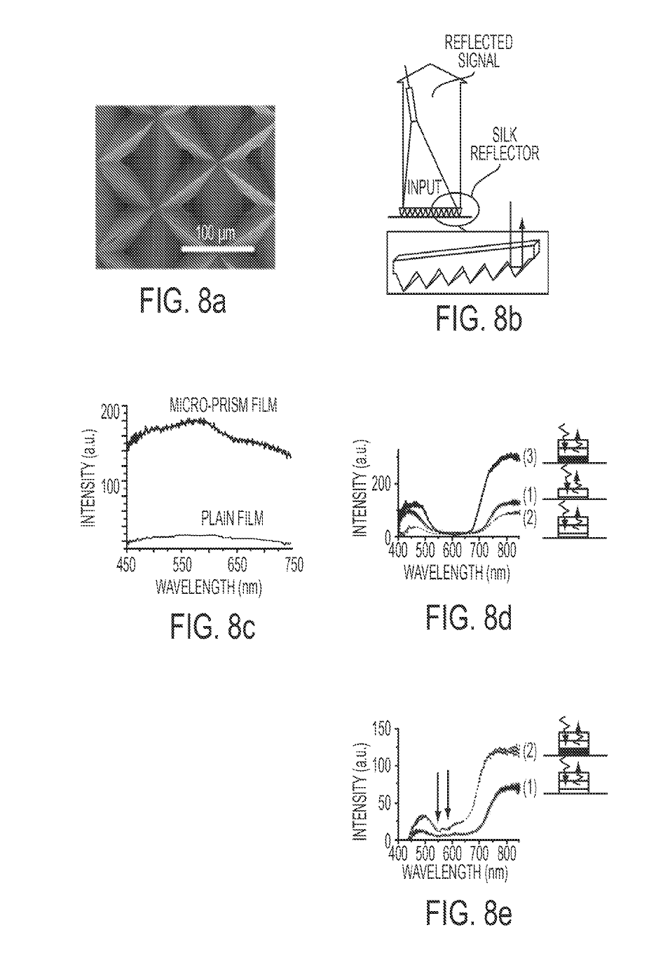

Silk MPAs were prepared by using micro-molding techniques akin to soft-lithography by replicating a micro-prism array master mask resulting in a 100 μm thick free-standing silk reflector film with dimensions up to tens of square centimeters (FIGS. 8A, 12 and 13).

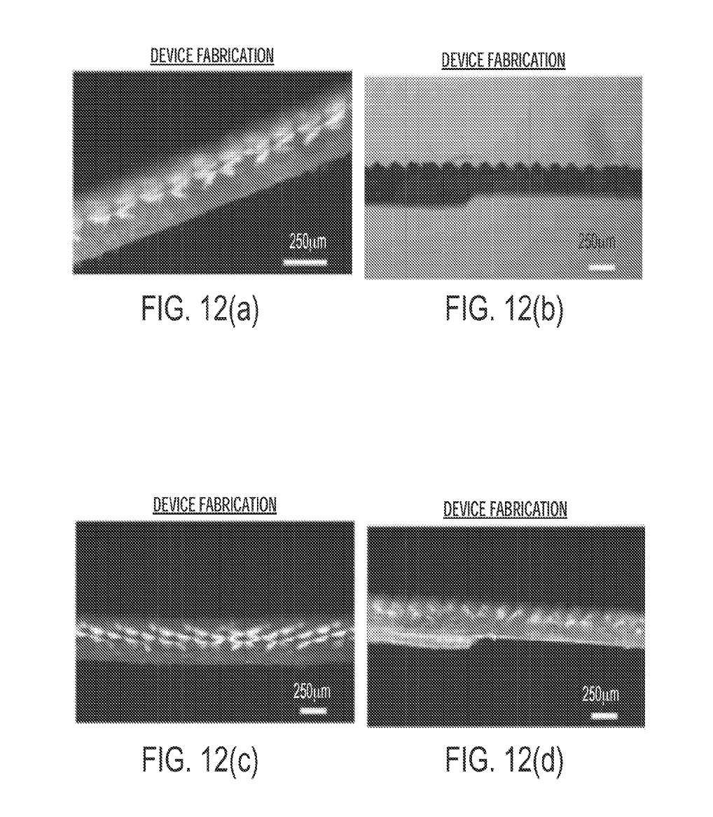

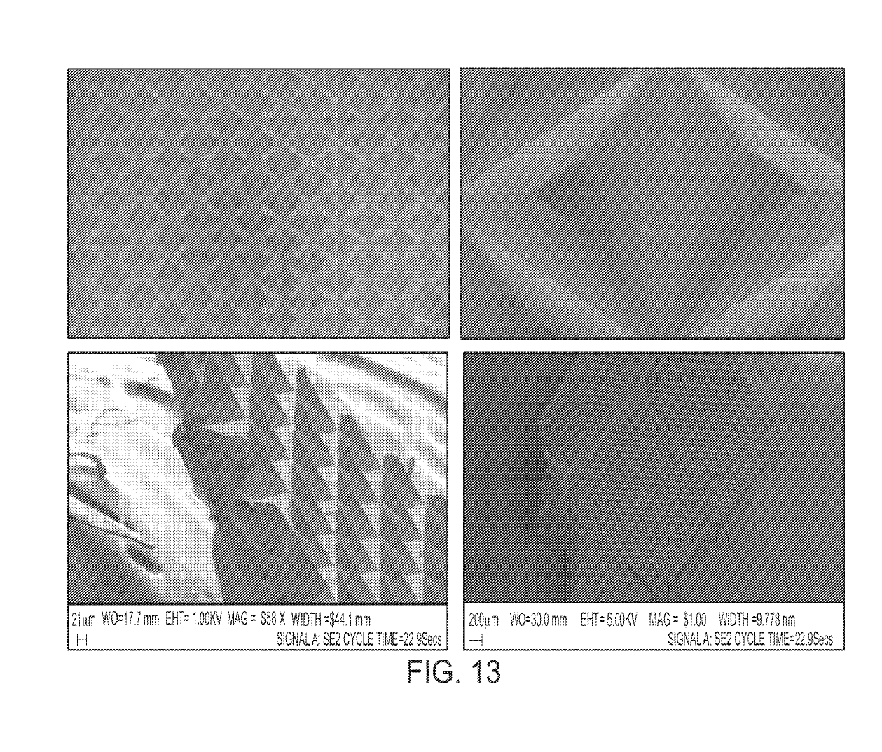

Silk based microprism reflectors, such as those shown in the micrographs provided in FIG. 12, may be fabricated using known methods. A water-based silk fibroin solution was obtained by extraction and purification of harvested Bombyx mori cocoons. This previously described process yields an 6.5-8% w/v silk fibroin solution which is then cast onto a microprism master mould (3M Scotchlite™ Reflective Material-High Gloss Film). The master consists of an array of microprisms with dimensions of roughly 100 micrometers and clustered in groups as shown in FIGS. 12 and 13. The silk is typically dried for 8-12 hours upon which it is mechanically detached from the master surface. Upon microscopic examination, the silk retroreflective films replicates the master and have a reflective appearance similar to the master mould. The index of refraction of silk is n=1.54.

Silk can be easily formed into mechanically robust films of thermodynamically-stable beta sheets, with control of thicknesses and surface feature sizes from just below ten nanometers to hundreds of micrometers or more. These films are formed by simple casting of purified silk solution which crystallizes upon exposure to air, without the need for exogenous cross-linking reactions or post processing cross-linking for stabilization.

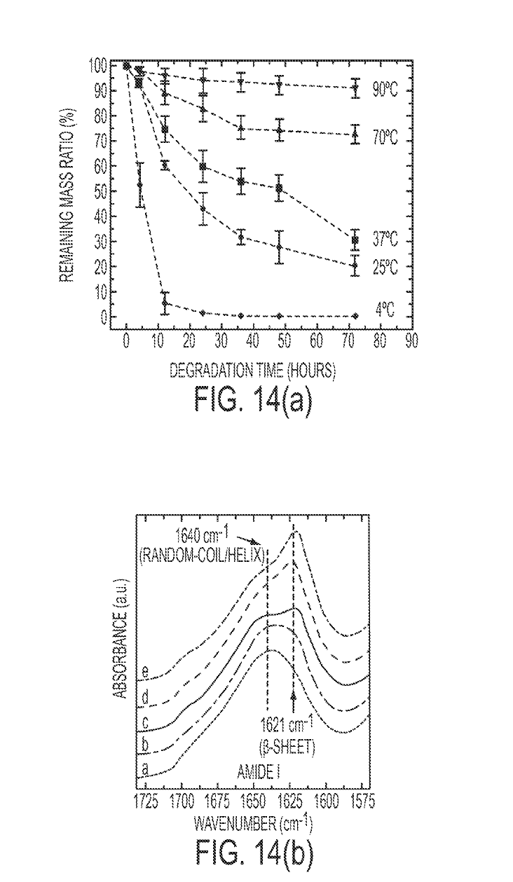

The dissolution rate of silk films is readily and controllably tunable, from instantaneous to years, via variation of the degree of crystallinity (β sheet content) introduced during material processing as shown in FIG. 14A (from Hu X, et al., “Microphase separation controlled beta-Sheet crystallization kinetics in fibrous proteins,” Macromolecules 2009; 42:2079 2087).

Different degrees of crystallinity can be assessed by FTIR spectroscopy as shown in FIG. 14B. This offers the possibility of creating structures and devices that are programmably degradable. Silk dissolution itself is mediated by chemical and biochemical processes such as enzymatic degradation.

The dissolution time of the MPA films can be tuned by controlling the degree of crystallinity during the silk protein self-assembly process by regulating the water content within the film through an annealing step. This approach can be used to allow rapid to slow degradation of the device depending on the application (FIG. 14). In the case of doxorubicin, drug delivery can be achieved in a localized and controlled fashion.

The utility of this passive optical device is to increase the amount of light that returns to a detector situated at the surface of a biological specimen when the reflector film is introduced underneath the specimen. An implantable silk MPA embedded in tissue could capture forward-scattered photons that are ordinarily lost in reflection-based imaging techniques. This would enhance intrinsic sensitivity for measurement over thicknesses where dimensions normally exceed typical photon mean free paths (MFP), absorption coefficient and scattering coefficient for most tissues, without resorting to coherent detection techniques or contrast agents for image enhancement. Not only does this performance allow for enhanced signal for deep tissue imaging, but should also allow for contrast enhancement, which is of even greater importance, since imaging of deep tissue malignancies is not necessarily limited by detection of light but rather contrast to the surrounding tissue. Hence, contrast enhancement is of great importance for improved diagnostics.

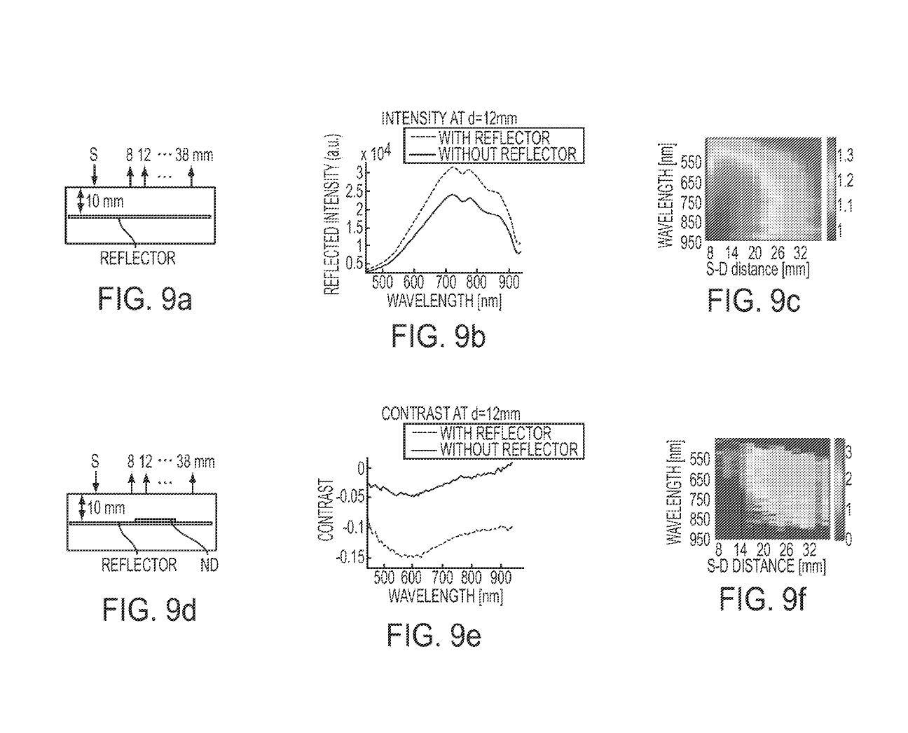

To validate the optical performance of the enhanced signal due to MPAs, diffuse reflected light from the silk MPAs was monitored under isotropic illumination of tissue like phantoms. For imaging of shallower depth, such as for subcutaneous applications, a co-localized source and detection unit was used (FIG. 8B). For imaging deeper tissue, the geometry of the fiber-based backscattering imaging setup (FIG. 9A) was such that a broadband light source was used for illumination and a detection fiber was scanned over the phantom, leading to illumination source—detector distances between 8 mm and 38 mm, in 2 mm increments, which allowed for probing multiple tissue depths.

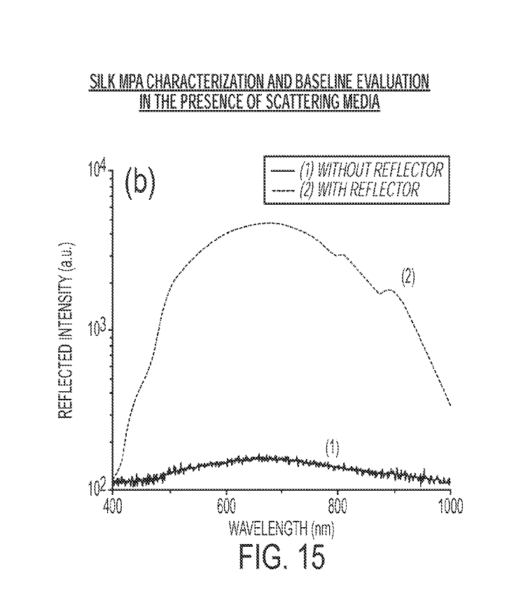

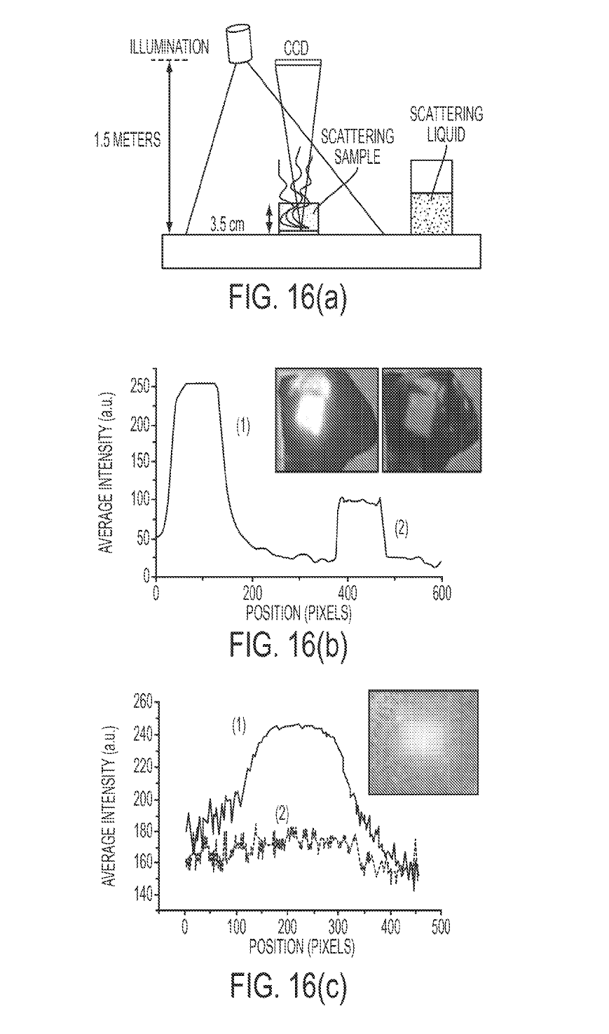

As shown, the presence of the reflector resulted in a significant enhancement of signal at the detector plane, increasing the backscattered signal intensity by nearly five-fold when compared to an unpatterned silk film (FIG. 8C) and by two orders of magnitude when compared with background (FIGS. 15 and 16).

Commonly used for safety applications, the performance of retroreflecting films is defined by measuring the luminous intensity and retroreflector coefficients per illuminance level on the surface of the retroreflector (in candelas/1× and candelas/(1×/m2), respectively). Designed for broad angle reflection in safety clothing and garments, the films have a reflection coefficient M (defined as the ratio of the coefficient of luminous intensity of a plane retroreflecting surface to its area expressed in candelas per square meter) between 300 and 400.

The silk reflector replicated the master faithfully and its optical performance matched the master's, providing orders of magnitude of measured increase in the diffuse reflection when compared to the background without any reflective surface (FIG. 15). For the device to be adaptable to biomedical environments, it must successfully integrate and operate in humid or wet scattering environments. Performance of the silk MPAs under these conditions was assessed by placing the silk reflector films under a 4 cm thick block of gelatin or submerged in a talcum powder and water suspension at a depth of 6.5 cm. In both cases the presence of the reflector resulted in a significant enhancement of signal at the detector plane, increasing the backscattered signal intensity in both cases and allowing easy imaging (with a commercial CCD camera) of the reflector under isotropic illumination (FIG. 16). It must be noted that, in contrast to the in-vivo and part of the in-vitro measurements, these baseline measurements are performed using the fiber probe at a distance from the scattering surface and not in contact with the scattering surface (in contrast, for example, with the in vivo and deep tissue experiments where the fiber probe is placed in contact with the skin).

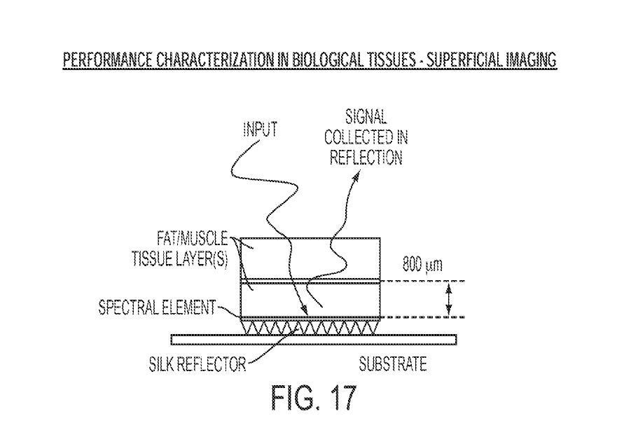

FIG. 17 illustrates an exemplary silk fibroin optical device. While the previous results establish a necessary baseline, in an optical diagnostic situation involving light scattering, it is important to acquire specific spectral information from the volume under test to associate it with physiological markers of interest. With this premise, two spectrally responsive elements embedded in biological tissue were used and an in-vitro experiment was performed to assess the variation in the optical response when the device was present. The silk reflector was placed underneath two types of spectral filters: a 10 nm bandpass multilayer filter (with central wavelength λ0=630 nm) and a layer of cellulose embedded with red pigment. These were chosen to provide known broadband and narrowband spectral responses to embed in tissue constructs to test the efficacy of the device. The reflector/spectral element was then covered either by single or multiple layers of 800-micrometer thick porcine fat or muscle tissue. The resulting structure was then probed by illumination with incoherent white light delivered through a multimode fiber. The latter is part of a fiber-backscattering probe which acts as the collector for the diffuse retroreflected scattering signal and redirects it to a spectrometer.

### Example 11

### Subcutaneous Applications

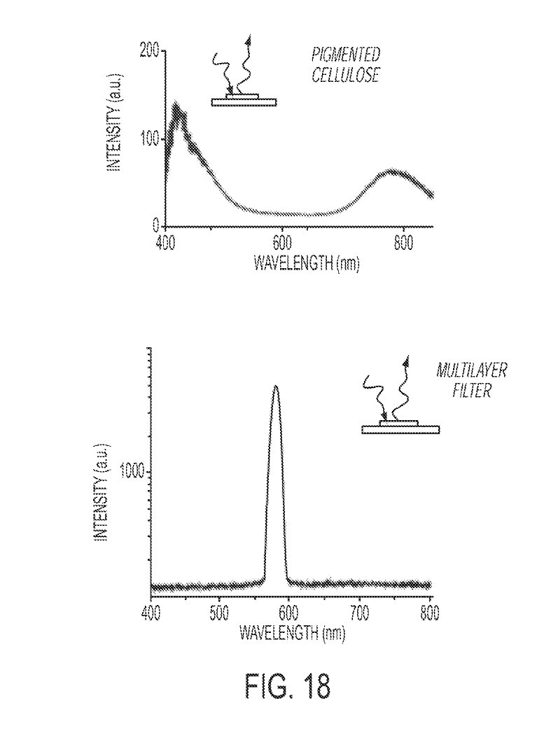

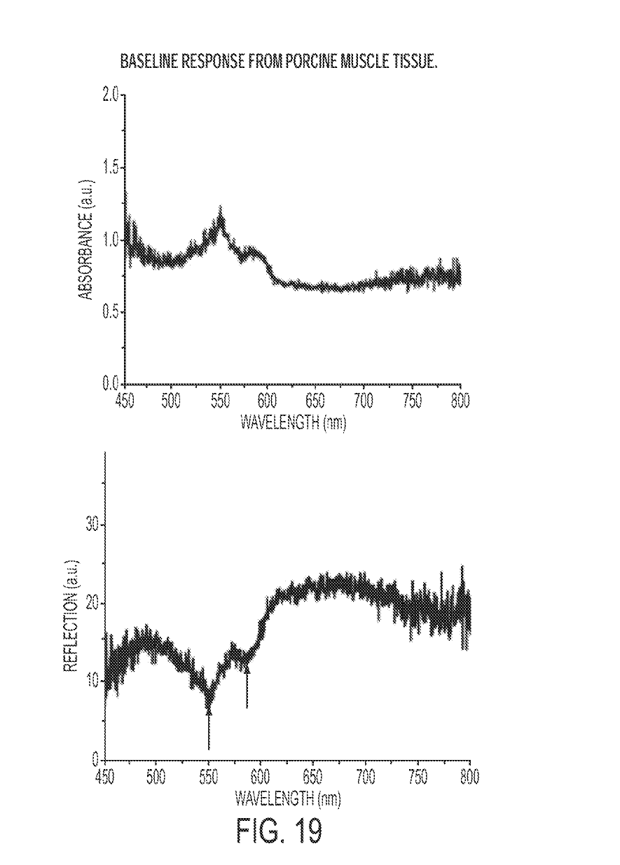

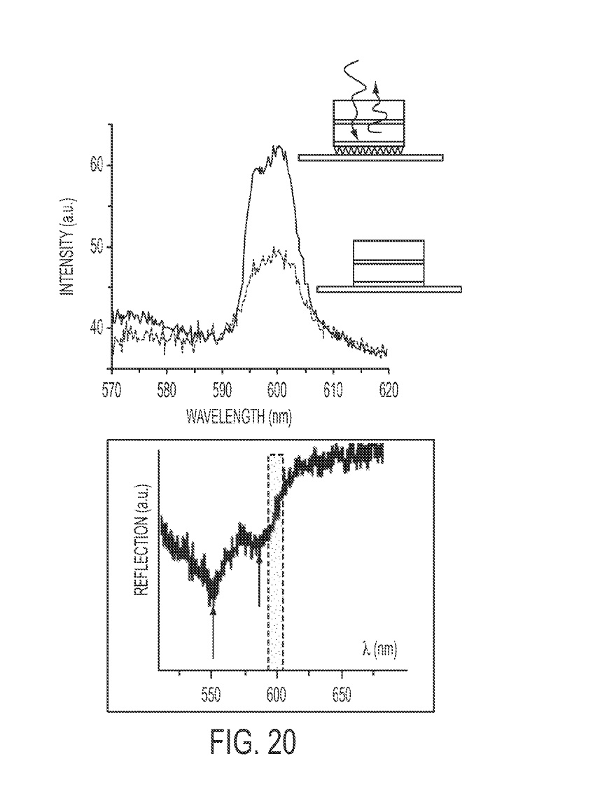

For subcutaneous applications (e.g., shallower depths), an in vitro experiment was performed to assess the variation in optical response caused by the presence of the MPA by placing the device under a layer of cellulose embedded with red pigment. The MPA reflector and cellulose combination was covered by single or multiple layers of 800-micrometer thick porcine fat (FIG. 8D) or by single or multiple layers of muscle tissue (FIG. 8E). In the absence of MPAs, the detected spectral response was progressively attenuated as layers of fat or muscle tissue were stacked on the device. Surprisingly, the presence of the silk reflector underneath the tissue structure significantly enhanced the backscattered signal collected and its dynamic range, allowing collection of the spectral response of the embedded pigmented layer (FIG. 18). A similar response was observed when using muscle tissue, where the presence of the silk MPA causes an increase in the dynamic range of the detected signal revealing the spectral signature of myocytes with absorption peaks appearing at λ1˜550 nm and λ˜2˜575 nm. Additional experiments are also presented in the FIGS. 19 and 20 (see below).

The Delrin phantom had a given thickness of 10 mm, the thickness of the liquid phantom was varied between 2 mm and 10 mm. The geometry of the imaging setup was such that a broadband (white) light source (halogen) was used for illumination and a detection fiber was scanned over the tissue, leading to source—detector positions between 8 mm and 38 mm, in 2 mm increments. The reflected signal was collected in a spectrometer setup. This scanning geometry was chosen in order to evaluate the spatial dependence of signal enhancement and also because it is well known that deeper, highly scattering tissue can only be imaged when there is a certain distance between the source and detector, where the distance is depth dependent. Imaging was performed on the phantoms for four different scenarios—Phantom alone, mirror embedded at the depth of interest, a 8 mm×8 mm neutral density (ND) filter (OD=0.6) piece on top of the mirror, and the ND without the mirror. The ND piece was used to mimic a local inclusion for evaluating the contrast enhancement. The location of the ND filter was ˜16 mm away from the source fiber in the x-y plane.

Signal enhancement was evaluated by imaging the phantom with and without the embedded mirror. The ratio between those two gives the enhancement in reflected intensity, hence signal enhancement. In the case of the solid phantom, where the mirror is embedded at 10 mm depth, we found that the signal could be enhanced 1.45 times in comparison to not having the mirror. This is a 45% enhancement at 10 mm depth of a highly scattering medium. For the liquid phantom without milk, a 1.25 times enhancement could be found. For the liquid phantom, where ink was used for mimicking absorption, a 15-20% enhancement could still be found at 10 mm depth. For shallower depths, this enhancement is even larger.

### Example 12

### Deep Tissue Applications

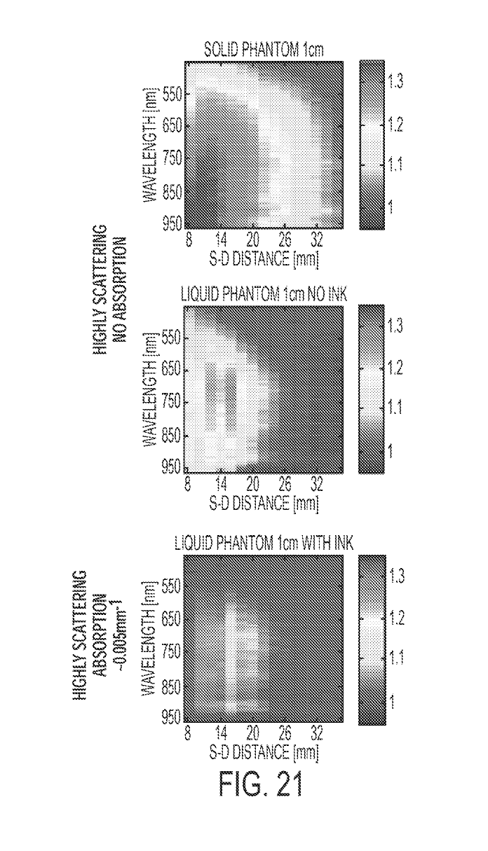

For demonstrating the potential for enhanced deep tissue imaging, experiments were performed on solid delrin phantoms (FIG. 9), which are highly scattering and mimic well the scattering in tissue, as well as liquid phantoms, which were made of a milk, water, and ink mixture, mimicking not only scattering, but absorption in tissue (FIG. 21).

For determining the signal enhancement at 1 cm depth, imaging was performed on the phantom alone and with the MPA embedded at 1 cm depth. The presence of the reflector inside the delrin phantom resulted in a significant enhancement of signal at the detector plane, increasing the backscattered signal intensity 1.4 times at source detector distances of ˜12 mm when compared to the phantom alone (FIG. 9B). The ratio between intensity with reflector and phantom alone can be seen in FIG. 9C for all source detector separations. Surprisingly, in the case of the liquid phantom (FIG. 21), which includes ink as an absorbing material, the enhancement is still significant (˜20%) even at 1 cm depth.

Optical imaging of malignancies has two major challenges—accessibility and contrast to healthy surrounding tissue. If the malignancy is too deep to be imaged, information content is lost. This limitation has been addressed in the previous paragraph, where we show that the signal can be enhanced even in 10 mm depth. The maybe even more important question is if contrast can be enhanced. For answering this question, we used a small piece of ND filter, mimicking an inclusion in tissue.

Contrast was defined as (I-I0)/I0, where I is the intensity measured with the ND being present and I0 without the ND filter (background signal). In the case of having the mirror embedded, I0 is the intensity reflected from the phantom with the mirror; in the case of no mirror, I0 is the intensity measured on the phantom alone.

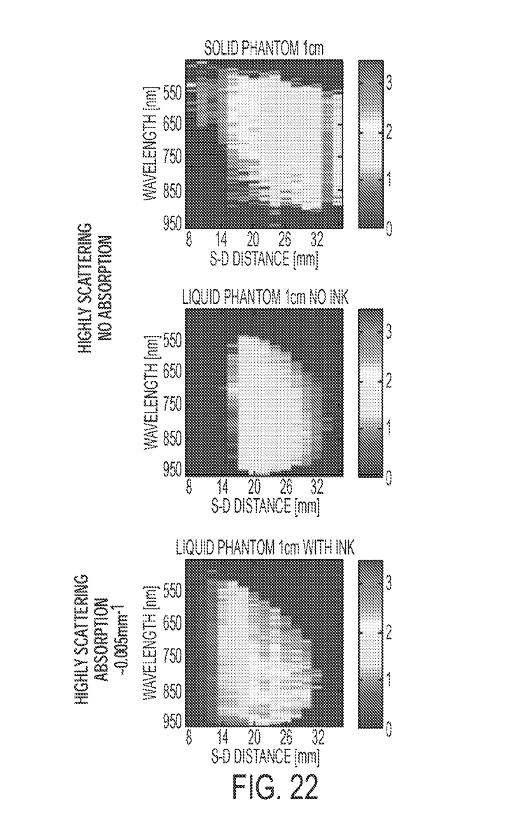

For the solid phantom, having the ND inclusion at 10 mm depth, we were able to see a ˜3 times bigger contrast with the mirror being present in comparison to not having the mirror. In the case of the liquid phantom without ink, a 3 times bigger contrast, and with ink a ˜1.5 times bigger contrast was found, hence a 50% increase in contrast when the mirror is present (see FIGS. 21 and 22).

Since diagnostics depend on differences between healthy and malignant tissue, contrast is of crucial importance. In order to determine contrast enhancement at 1 cm depth, an 8 mm×8 mm neutral density (ND) filter (OD=0.6) was used to mimic a local inclusion (FIG. 9D).

Imaging was performed with the ND filter on top of the reflector as well as with the ND filter alone. Contrast was defined as the (I-I0)/I0, where I is the measured reflected intensity with the ND filter at 1 cm, I0 is the background intensity without the reflector. A 3.5 times increase in contrast was found (FIG. 9E) at source detector distance of 12 mm. The contrast enhancement for all source detector distances can be found in FIG. 9F. While contrast enhancement in 1 cm depth of the liquid phantom (FIG. 22) was reduced in comparison to the delrin phantom, it was still 2.5 times larger in comparison to no embedded reflector.

### Example 13

### In Vivo Applications

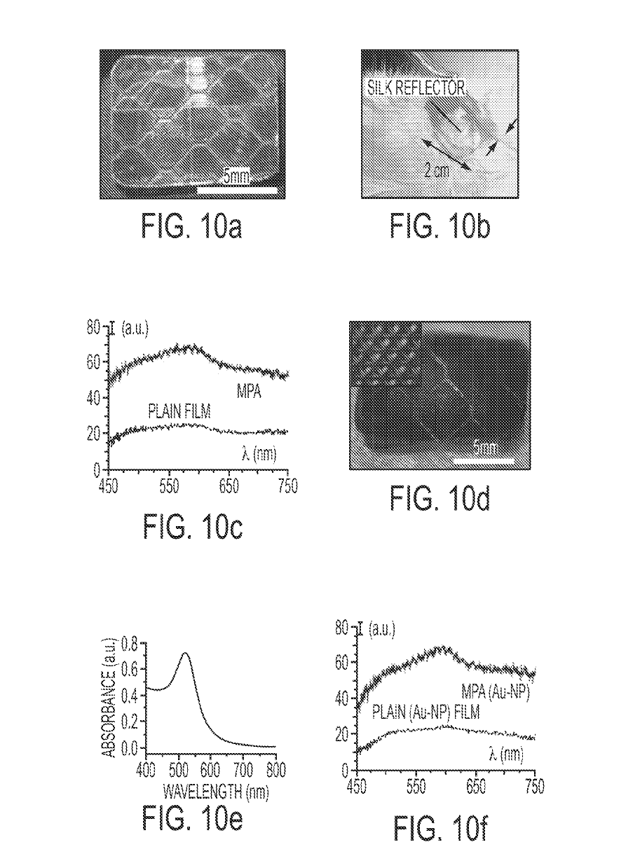

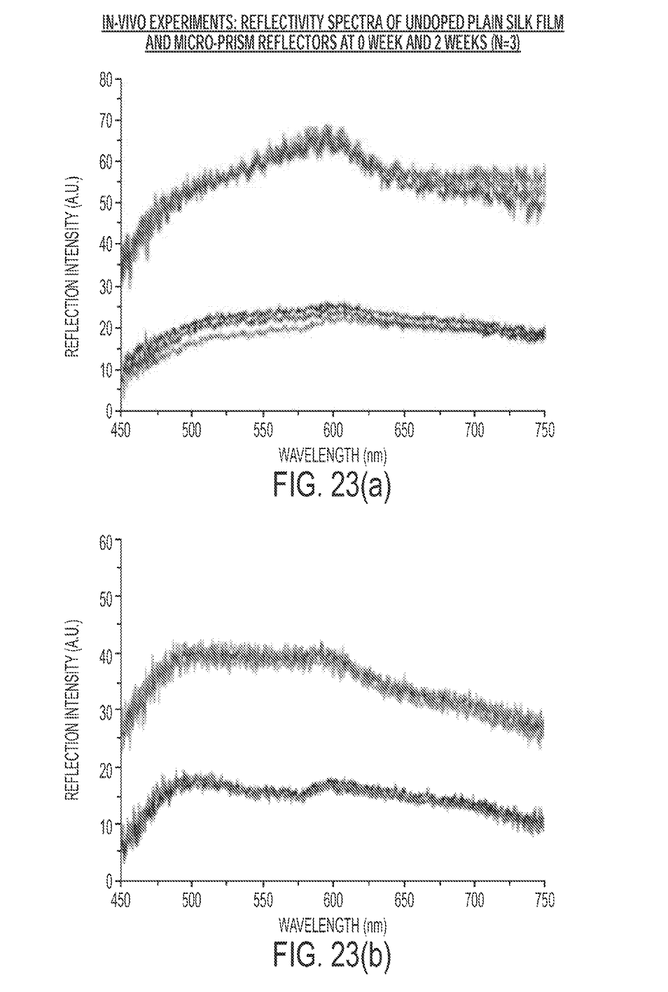

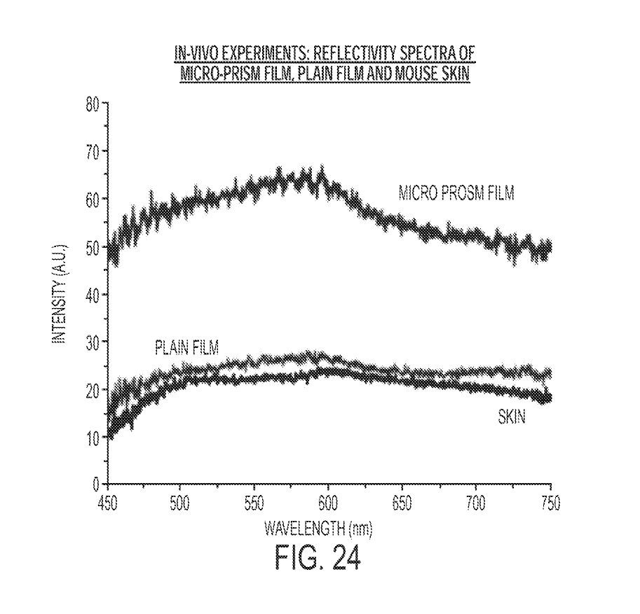

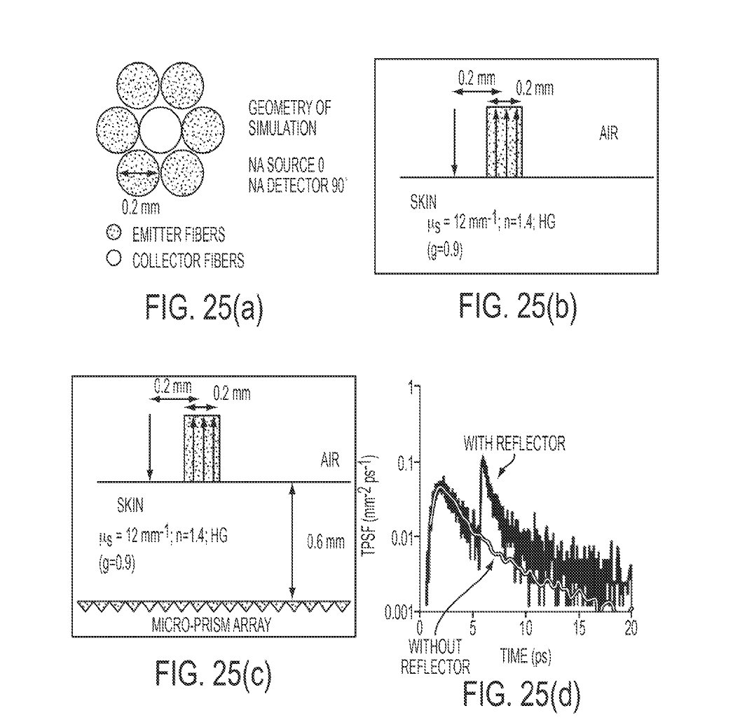

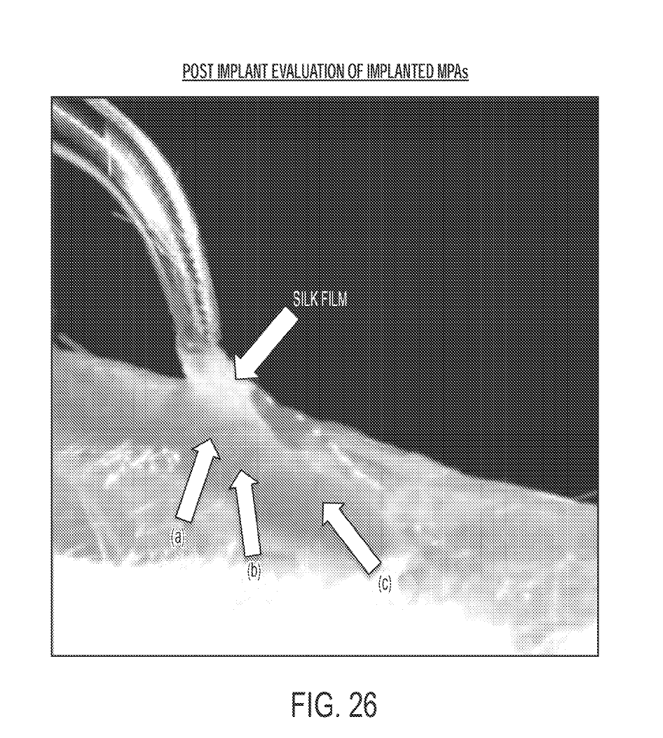

These in vitro results provided initial validation of the silk MPA performance in an in vitro environment and its utility for improved imaging. To support the applicability of the concept as an implantable device, in vivo studies were conducted by implanting the silk MPA structures in Balb/c mice (FIGS. 10A and 10B) in accordance with institutional IACUC-approved protocols. Two samples, one flat silk and one micro-patterned MPA silk film (both 100 micrometer thick, ˜1 cm×1 cm) were inserted subcutaneously after ethylene oxide sterilization through an incision on the back of the mice. After suturing the wound site, the scattered signal was measured with the colocalized imaging geometry (N=3, FIG. 23). The backscattered illumination through the mouse skin was collected by a fiber probe at the implant site, and a three-fold improvement (FIG. 10C) in collected signal was measured with the MPA in comparison to the control areas (mouse skin where either a flat film or no film was present) (FIG. 24).

Further substantiation of the MPA performance was carried out using a Monte Carlo code to solve the radiative transfer equation, an integro-differential equation widely used for describing light propagation in random media such as biological tissues. The approach was used to calculate the backscattered signal intensity in the presence of the silk MPA reflector.

In this simulation, the silk device was postulated to provide 100% reflectivity and to be located at a depth of 0.6 mm under the skin surface with a scattering coefficient of μs=12 mm-1 and an absorption coefficient of μa=0.01 mm-1, typical of skin and muscle tissues in the NIR

wavelength range (650-850 nm), showing a predicted increase in reflected signal in agreement

with what is observed experimentally (˜3× reflectivity enhancement) (FIG. 25).

### Example 14

### Post-Implantation Reflector Evaluation

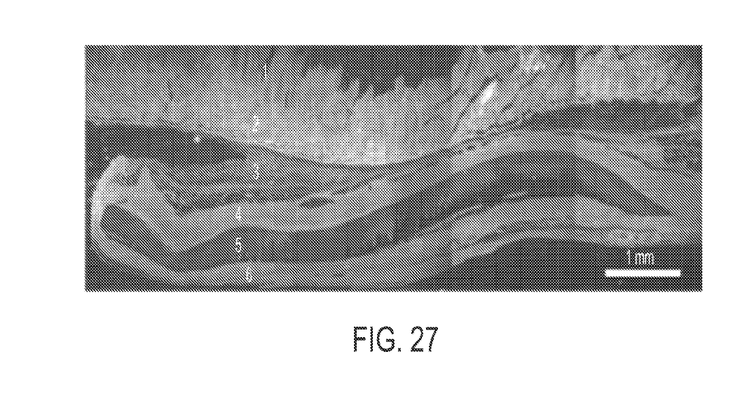

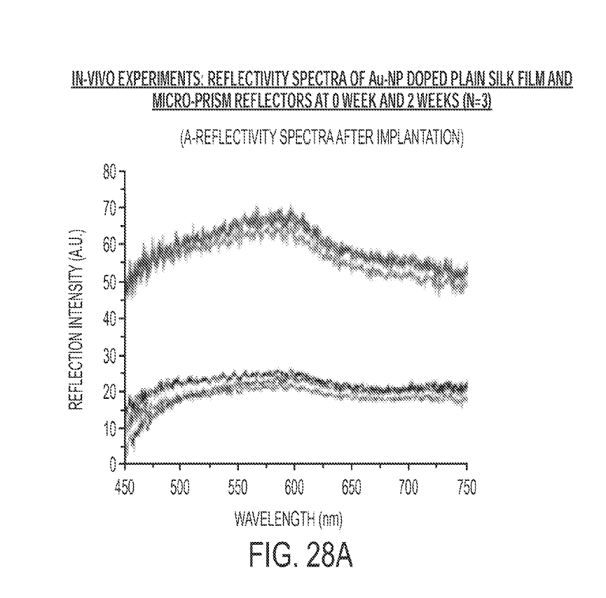

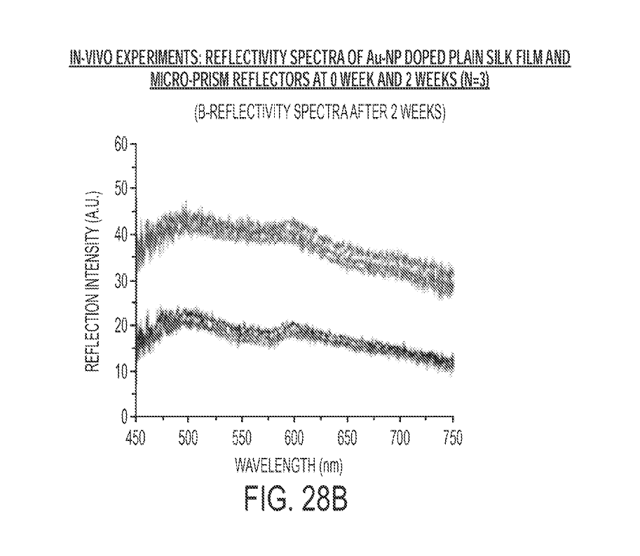

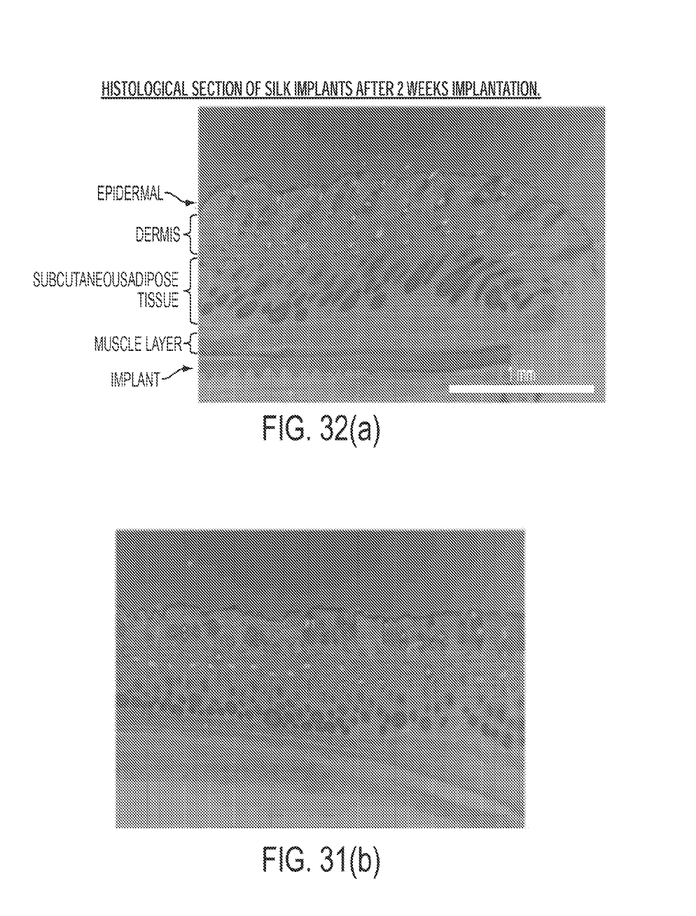

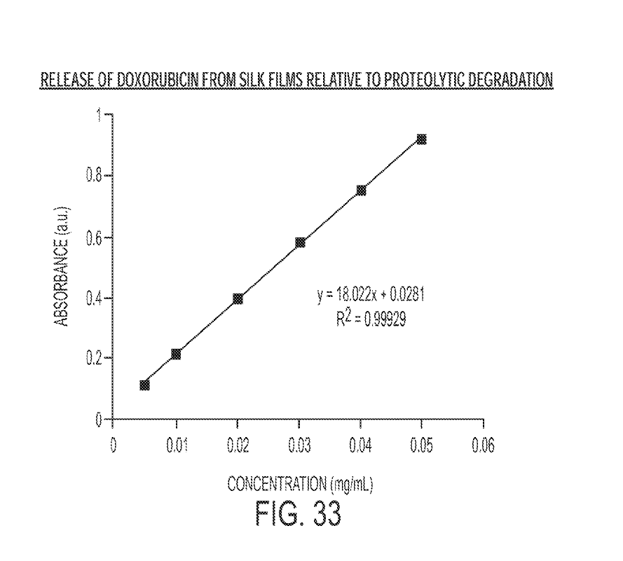

The reflector performance was monitored in the same mice two weeks after implantation. The measured signal enhancement was found to be lower than the initial value (˜2× reflectivity enhancement, FIG. 28) because of enzymatic degradation and initial remodeling and reintegration of the MPA in the native tissue, as designed. This process directly affects the optical quality of the implant. The devices were also monitored for adverse reactions and resorbability by histopathological sections of the implanted silk film and the underlying tissue. No visible inflammation was found at 2 weeks after implantation. Moreover, initial evidence suggested re-incorporation of the implanted device into the tissues. For example, revascularization on the surface of the film (FIG. 27) was observable upon examination of flat films after 4 weeks of implantation by examining the excised tissue. It was also still possible to identify the micro-prism arrays in the histological sections (FIGS. 27 and 32).

### Example 15

### Operational Multifunctionality

Whereas the optical utility alone provides a demonstration of in vivo integration of these optical devices within living tissue and improved imaging capabilities, additional advantages are present at the union of form and function enabled by the silk biomaterial platform with incorporated dopants. This integrate of active moieties generates doped silk MPAs with additional biomedical relevance beyond enhanced optical imaging. In order to demonstrate that the imaging capabilities of such functionalized MPA can be maintained, silk MPAs were prepared with a silk Au-NP solution. The Au-NPs, prepared according to published protocols20, were mixed in the silk solution, which was subsequently cast on the previously used micro-prism masters, yielding free-standing Au-NP-silk MPAs (FIG. 10D). Similarly to gold nanoshells (Au-NSs), Au-NPs resonantly absorb specific wavelengths (FIG. 10E) of incident light and convert this energy to heat. This technique has been successfully used in phototherapy for in vivo medical applications, such as to treat infections, tumor mitigation and pain relief.

The resulting Au-NP silk micro-prism reflectors were implanted in mice, alongside control Au-NP-doped flat films, following the same procedures previously described.

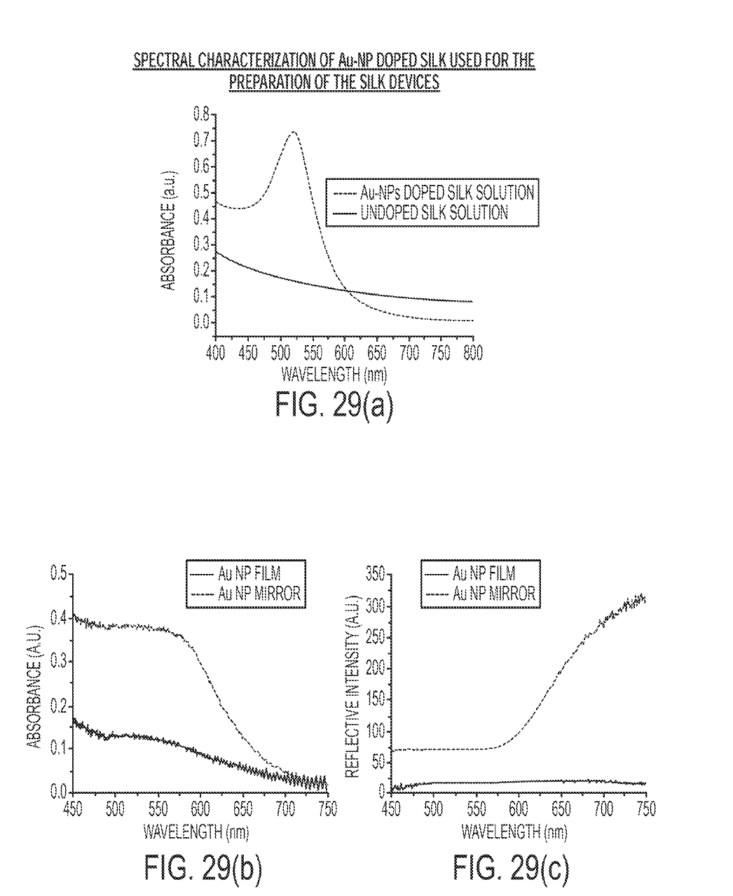

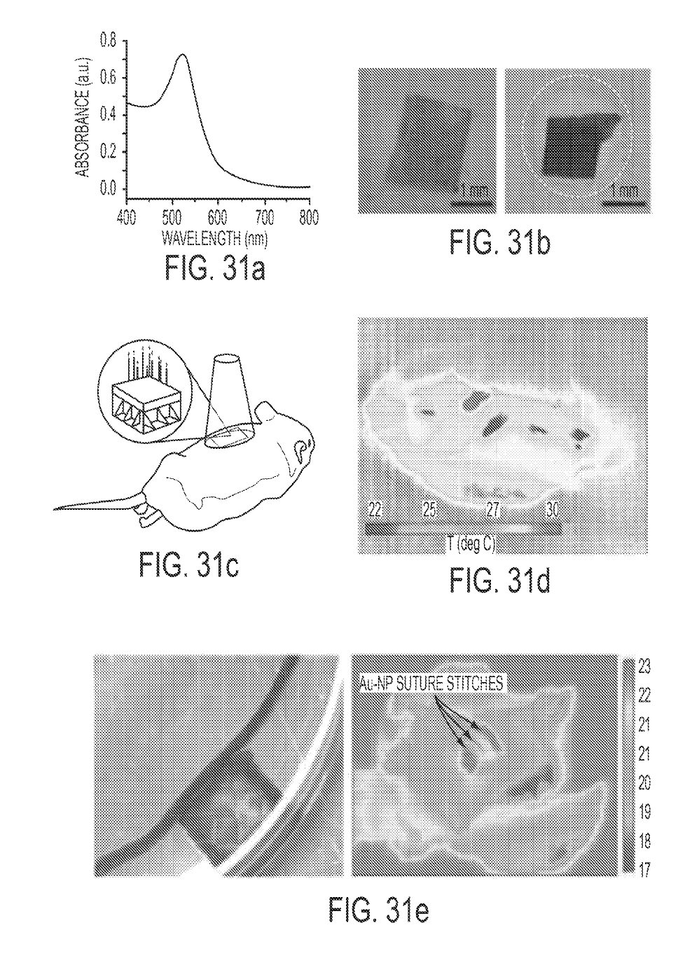

The in vivo measurements at t=0 weeks (FIG. 10F) and t=2 weeks (FIG. 28) after implantation display similar optical performance, which is 3 times signal enhancement compared to plain film, to what was previously observed for the undoped devices by enhancing the diffuse reflective signal (N=3, FIG. 28). Hence, the doped film does not change the bulk signal enhancement. However, a detectable difference in the in vitro spectral response caused by the absorption of the Au-NPs entrained within the silk matrix (FIG. 29) was found. Since the absorption spectra is changed in comparison to a plain film and is specific to the Au-NP, the doped MPA become functionalized, and the optical performance of the device can be tuned by the Au-NP-MPAs localized light absorbing patches. This can be demonstrated by illuminating the mice with green laser light to match the absorption peak of the Au-NPs entrained in the film (FIG. 31). A green laser beam (Coherent Verdi 10) of initial diameter w 0=3 mm is expanded to a diameter of w˜5 cm, corresponding to an irradiance of ˜0.13 W/cm2 to cover the back of the Balb-c mouse.

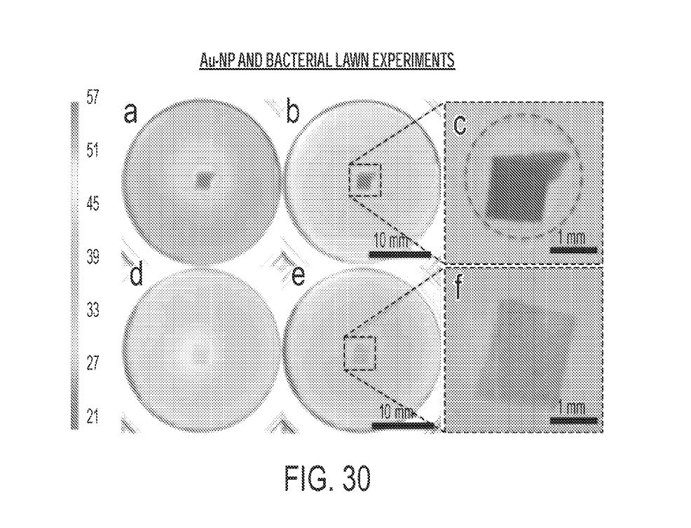

A thermal image (FLIR model SC645) of the mouse shows an area of increased temperature (ΔT˜5° C.) at the implant site corresponding to the subcutaneous Au-NP mirror (FIG. 31). This localized temperature increase is also used to demonstrate in vitro the elimination of bacteria by placing Au-NP-MPAs in contact with a bacterial lawn and illuminating with green light (FIGS. 30 and 31). For the in vivo samples, as before, histopathological sections of the Au-NP-MPAs and Au-NP doped films revealed no inflammatory response, encapsulation or fibrosis after 2 weeks of implantation (FIG. 32). In the case of the silk-AuNP-MPA, optical enhancement and plasmon absorption from the Au-NP provide independent functions within the same implantable device without reducing the functionality of either.

The work described herein presents a promising opportunity for such doped MPA is to add therapeutic functionality and have the entrained dopant modulate optical performance of the device. Specifically, the invention encompasses combining the capacity of silk materials to stabilize entrained labile compounds by co-locating them in the same device. This device can provide drug stabilization and controlled drug delivery, while simultaneously providing an optical feedback of delivery.

In general, common approaches for drug delivery can be roughly divided into two groups. The first group consists of small scale implantable systems for sustainable, long term drug release, which most commonly lack the ability to provide feedback of drug delivered. The second group focuses on targeted drug delivery, for example, with functionalized nanoparticles, where the drug delivery mechanism is a burst release. In the latter case, optical monitoring of drug release can be achieved by triggering not only the release (e.g., binding to the cell surface receptor) but a fluorescent marker. In comparison, the approach presented here provides advantage over more conventional approaches for drug delivery discussed above. For example, no additional compound is required for triggering of the release, since the degradation of the MPA itself is controlling the release. Moreover, the invention described herein makes it possible to release a drug in a sustainable, long term manner as well. Importantly, the optical performance of the device should degrade with the degradation of the device, hence allowing for quantification of drug release in real time over the full time period of release without the need for auxiliary device.

### Example 16

### Silk Fibroin Delivery Matrix with Doxorubicin

Hence, the implantable optical device embraced by the present application can serve its therapeutic function, while changes in reflectivity would be related to the amount of drug eluted and used, as a drug delivery monitoring mechanism. To this end, we used as a model system doxorubicin (DxR), a therapeutic dopant commonly used in the treatment of a wide range of cancers, including many types of carcinomas and soft tissue sarcomas.

DxR was added to the silk solution which was then reformed into free-standing DxR-silk micro-prism films. The performance of the silk-DxR-MPA was evaluated in vitro to correlate the quantity of eluted drug with changes in reflectivity of the MPA. The silk DxR-MPAs were immersed in a broad spectrum serine proteinase solution (proteinase k with a concentration of 0.1 mg/mL, FIG. 3 & Table. 3 shown above) to mimic the degradation process that the devices would be subject to in an in vivo environment.

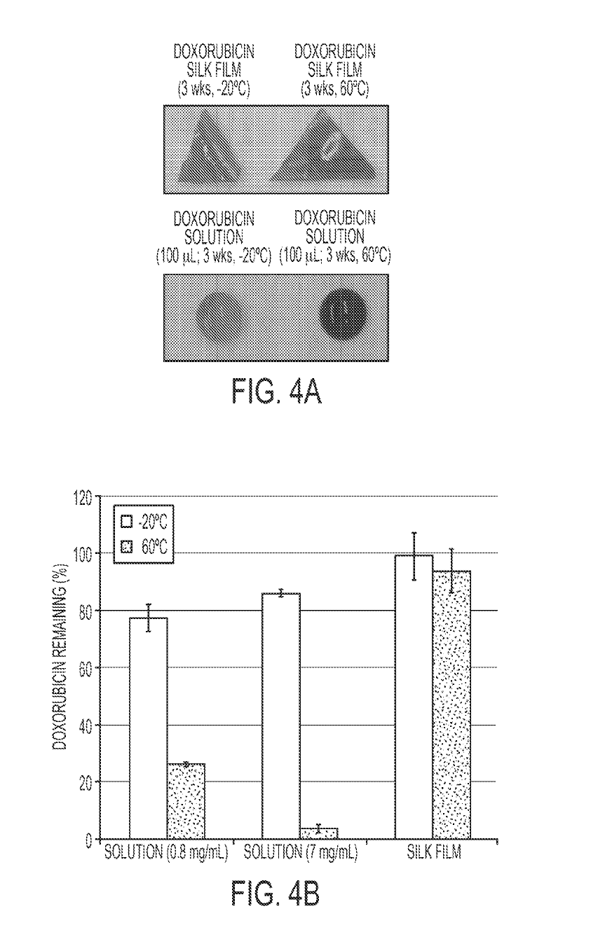

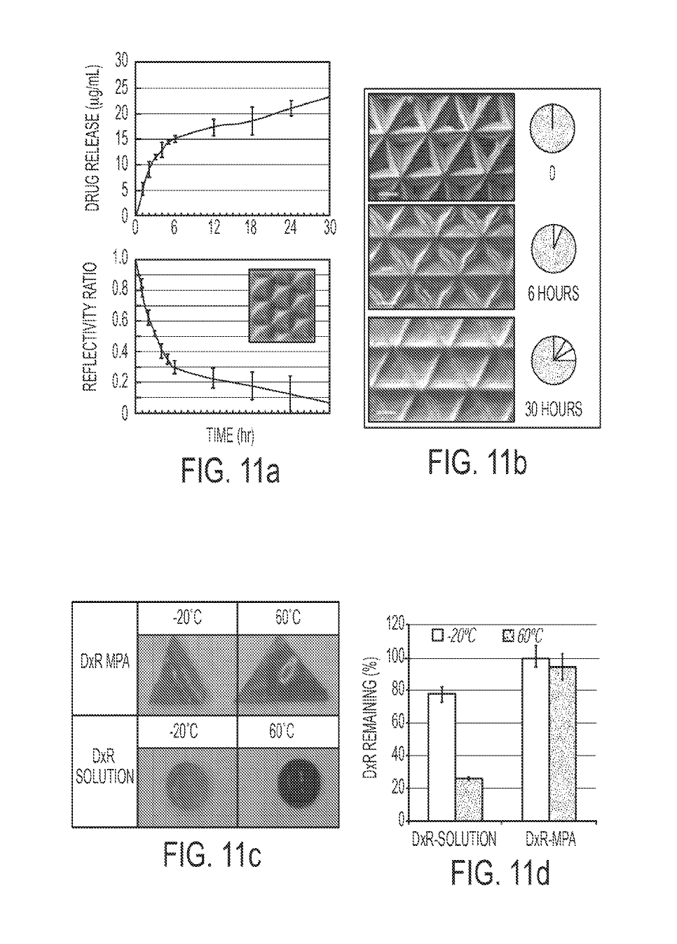

The silk DxR-MPA was evaluated at different time points for optical performance and drug content eluted by measuring absorbance at 495 nm and comparing measured values to a standard curve (FIGS. 11A & 33). The resulting curves were compared to the reflectivity of the silk-DxR-MPA at different time points, to show the relationship between drug released and the measured MPA reflectivity (with a correlation coefficient ρ=0.98). The changes in reflectivity from the MPAs follow both the burst release and the sustained release phase. The drug release in this case was predominantly degradation-mediated (e.g., drug only releases as the matrix degrades). The signal decrease as a function of micro-prism degradation also substantiates previous observations from the undoped silk-MPAs and is further corroborated by inspecting the silk-DxR-MPA at different stages of incubation in the proteinase buffer (0 hour, 6 hours, 30 hours) that show the degradation of the micro-prism structure (FIG. 11B). Additionally, these devices possess the ability to store and maintain the efficacy of doxorubicin. The doxorubicin-loaded silk-MPAs were stored at −20° C. (frozen) and 60° C. for 3 weeks after which it was determined that the fluorescence of the doxorubicin stored in silk films did not significantly decrease, despite the 80° C. range in storage temperature, in relation to comparable samples in solution (FIGS. 11C, 11D and 4). These results demonstrate the multi-functionality of the device, from optical enhancement for imaging, to drug storage, to optical monitoring and quantification of drug delivery in real time and over the entire time period of release.

The work presented in the present application enables new a class of optical devices that becomes fully integrated into regenerated tissues over time once their diagnostic and/or therapeutic utility is exhausted, eliminating the need for retrieval and extending the utility of in vivo screening modalities. Silk proves to be a particularly favorable material for these needs because it provides a convenient connection of material form and biological function. The ability to control the material degradation properties and reform silk biopolymers into technological formats adds opportunity for devices that can seamlessly operate with multifunctionality, that is, at the nexus of diagnostics (through optical transduction), therapy (through drug stabilization and delivery), and/or quantitative feedback of therapy (through drug delivery imaging within the same device).

The work presented here bears particular promise given the implications of individualized monitoring of drug delivery in vivo and the concepts for multifunctional devices, where a single device can administer a cure, while providing information of disease progression. It is further contemplated that the utility of multifunctional bioresorbable devices goes beyond medical applications into environmental monitoring or food safety where such devices could be used without negative impact on the environment or the consumer.

### REFERENCES

Shuang Cai, Sharadvi Thati, Taryn R. Bagby, Hassam-Mustafa Diab, Neal M. Davies, Mark S. Cohen, M. Laird Forrest. Localized doxorubicin chemotherapy with a biopolymeric nanocarrier improves survival and reduces toxicity in xenografts of human breast cancer. Journal of Controlled Release 146 (2010) 212-218.
Pritchard E M, Hu X, Finley V, Kuo C K, Kaplan D L. Effect of Silk Protein Processing on Drug Delivery from Silk Films. 2011, about to be submitted
Kevin A. DaSilva, James E. Shaw, JoAnne McLaurin. Amyloid-β fibrillogenesis: Structural insight and therapeutic intervention. Exp Neurol. 2010 223(2) 311-21.
Andreas Lammel, Xiao Hu, Sang-Hyug Park, David L. Kaplan, Thomas Scheibel. Controlling silk fibroin particle features for drug delivery. Biomaterials. 2010 31(16) 4583-4591.
Askansas V, Alvarez R B, Engel W K. beta-Amyloid precursor epitopes in muscle fibers of inclusion body myositis. Ann. Neurol. 1993 34(4) 551-60
Docampo, R.; Moreno, S. N. (1990), “The metabolism and mode of action of gentian violet”, Drug Metab. Rev. 22 (2-3): 161-178
M Tanihara, Suzuki Y, Nishimura Y, Suzuki K, Kakimaru Y, Fukunishi Y. A Novel Microbial Infection-Responsive Drug Release System. (1999) 88(5): 510-514
Suzuki, Y.; Tanihara, M.; Nishimura, Y.; Suzuki, K.; Kakimaru, Y.; Shimuzu, Y. A novel wound dressing with an antibiotic delivery system stimulated by microbial infection. ASAIO J. 1997, 43, M854-M857
Benedict Law, Ching-Hsuan Tung. Proteolysis: A Biological Process Adapted in Drug Delivery, Therapy, and Imaging. Bioconjugate Chem., Vol. 20, No. 9, 2009 1683-1695.
Bandak S, Ramu A, Barenholz Y, Gabizon, A. (1999) Reduced UV-Induced Degradation of Doxorubicin Encapsulated in Polyethyleneglycol-Coated Liposomes. Pharmaceutical Research. 16(6) 841-846.
Wood M J, Irwin W J, Scott D K. Photodegradation of doxorubicin, daunorubicin and epirubicin measured by high-performance liquid chromatography. J. Clin. Pharm. Ther. (1990) 15(4)291-300
Law S L, Chiang C H, Lin F M, The G W. Effect of stabilization temperature on the degradation of adriamycin in albumin microspheres. Biomater. Artif. Cells Immobilization Biotechnol. (1991) 19(3):613-629.
Howard, D. et al. Immunoselection and adenoviral genetic modulation of human osteoprogenitors: in vivo bone formation on PLA scaffold. Biochemical and Biophysical Research Communications 299, 208-215 (2002).
Partridge, K. et al. Adenoviral BMP-2 gene transfer in mesenchymal stem cells: In vitro and in vivo bone formation on biodegradable polymer scaffolds. Biochemical and Biophysical Research Communications 292, 144-152 (2002).
Stone, K. R., Steadman, J. R., Rodkey, W. G. & Li, S. T. Regeneration of meniscal cartilage with use of a collagen scaffold—Analysis of preliminary data. Journal of Bone and Joint Surgety-American Volume 79A, 1770-1777 (1997).
Kim, D. H. et al. Dissolvable films of silk fibroin for ultrathin conformal bio-integrated electronics. Nat Mater 9, 511-517 (2010).
Parker, S. T. et al. Biocompatible Silk Printed Optical Waveguides. Advanced Materials 21, 2411-+(2009).
Altman, G. H. et al. Silk-based biomaterials. Biomaterials 24, 401-416 (2003).
Amsden, J. J. et al. Rapid Nanoimprinting of Silk Fibroin Films for Biophotonic Applications. Advanced Materials 22, 1746-+(2010).
Lawrence, B. D., Cronin-Golomb, M., Georgakoudi, I., Kaplan, D. L. & Omenetto, F. G. Bioactive silk protein biomaterial systems for optical devices. Biomacromolecules 9, 1214-1220 (2008).
Perry, H., Gopinath, A., Kaplan, D. L., Dal Negro, L. & Omenetto, F. G. Nano- and micropatterning of optically transparent, mechanically robust, biocompatible silk fibroin films. Advanced Materials 20, 3070-3072 (2008).
Omenetto, F. G. & KapLan, D. L. A new route for silk. Nature Photonics 2, 641-643 (2008).
Wilz, A. et al. Silk polymer-based adenosine release: Therapeutic potential for epilepsy. Biomaterials 29, 3609-3616 (2008).
Pritchard, E. M. & Kaplan, D. L. Silk fibroin biomaterials for controlled release drug delivery. Expert Opinion on Drug Delivery 8, 797-811 (2011).
Szybala, C. et al. Antiepileptic effects of silk-polymer based adenosine release in kindled rats. Experimental Neurology 219, 126-135 (2009).
Jin, H. J. et al. Water-stable silk films with reduced beta-sheet content. Adv Funct Mater 15, 1241-1247 (2005).
Lu, Q. et al. Water-insoluble silk films with silk I structure. Acta Biomater 6, 1380-1387 (2010).
Hu, X. et al. Regulation of Silk Material Structure by Temperature-Controlled Water Vapor Annealing. Biomacromolecules 12, 1686-1696 (2011).
Matcher, S. J., Cope, M. & Delpy, D. T. In vivo measurements of the wavelength dependence of tissue-scattering coefficients between 760 and 900 nm measured with time-resolved spectroscopy. Appl Optics 36, 386-396 (1997).
Zonios, G. & Dimou, A. Light scattering spectroscopy of human skin in vivo. Opt Express 17, 1256-1267 (2009).
Ishimaru, A. Wave-Propagation and Scattering in Random-Media and Rough Surfaces. P Ieee 79, 1359-1366 (1991).
Liang, Z. Q. et al. A centrifugation-based method for preparation of gold nanoparticles and its application in biodetection. Int J Mol Sci 8, 526-532 (2007).
Huang, W. C., Tsai, P. J. & Chen, Y. C. Functional gold nanoparticles as photothermal agents for selective-killing of pathogenic bacteria. Nanomedicine-Uk 2, 777-787 (2007).
O′ neal, D. P., Hirsch, L. R., Halas, N. J., Payne, J. D. & West, J. L. Photo-thermal tumor ablation in mice using near infrared-absorbing nanoparticles. Cancer Lett 209, 171-176 (2004).
Jaeger, G. T., Larsen, S., Soli, N. & Moe, L. Two years follow-up study of the pain-relieving effect of gold bead implantation in dogs with hip joint arthritis. Acta Vet Scand 49 (2007).
Domachuk, P., Perry, H., Amsden, J. J., Kaplan, D. L. & Omenetto, F. G. Bioactive “self-sensing” optical systems. Appl Phys Lett 95 (2009).
LaVan, D. A., McGuire, T. & Langer, R. Small-scale systems for in vivo drug delivery. Nat Biotechnol 21, 1184-1191 (2003).
Liu, Y., Miyoshi, H. & Nakamura, M. Nanomedicine for drug delivery and imaging: a promising avenue for cancer therapy and diagnosis using targeted functional nanoparticles. Int J Cancer 120, 2527-2537 (2007).
Kim, M. et al. Real-time monitoring of anticancer drug release in vitro and in vivo on titania nanoparticles triggered by external glutathione. Talanta 88, 631-637 (2012).
Weinstain, R., Segal, E., Satchi-Fainaro, R. & Shabat, D. Real-time monitoring of drug release. Chem Commun (Camb) 46, 553-555 (2010).
Wang, Y. et al. In vivo degradation of three-dimensional silk fibroin scaffolds. Biomaterials 29, 3415-3428 (2008).

### Other Embodiments

It should be understood that this invention is not limited to the particular methodology, protocols, and reagents, etc., described herein and as such may vary. The terminology used herein is for the purpose of describing particular embodiments only, and is not intended to limit the scope of the invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

1. A method for local delivery of a therapeutic agent in a subject, the method comprising a step of:
administering to a subject in need of a therapy for treating a condition
a composition comprising a silk fibroin matrix and a therapeutic agent incorporated therein,
wherein the composition is implanted in the subject, such that the therapeutic agent is locally released from the silk fibroin matrix over a period of time in an amount effective to treat the condition.

2. The method of claim 1, wherein the subject has at least one localized lesion.

3. The method of claim 2, wherein the composition is positioned at or near a site of the localized lesion in the subject.

4. The method of claim 2, wherein the localized lesion is a site of surgical procedure.

5. The method of claim 4, wherein the surgical procedure comprises a biopsy.

6. The method of claim 4, wherein the surgical procedure comprises a transplant.

7. The method of claim 4, wherein the surgical procedure comprises a repair or reconstruction of a tissue.

8. The method of claim 4, wherein the surgical procedure comprises resection of a diseased or injured tissue.

9. The method of claim 4, wherein the surgical procedure comprises resection of a tumor, a lymph node, or combination thereof.

10. The method according to claim 1, wherein the therapeutic agent is an anti-cancer agent, an antibiotic agent, an immunomodulatory agent, a mitogenic agent, a regulator of an extracellular matrix (ECM), an enzyme, or any combination thereof.

11. The method of claim 10, wherein the anti-cancer agent is selected from the group consisting of a chemotherapeutic agent, a cytotoxic agent, radioisotopes, toxins, enzymes, enzymes to activate prodrugs, radio-sensitizing drugs, interfering RNAs, superantigens, anti-angiogenic agents, alkylating agents, purine antagonists, pyrimidine antagonists, plant alkaloids, intercalating antibiotics, aromatase inhibitors, anti-metabolites, mitotic inhibitors, growth factor inhibitors, cell cycle inhibitors, enzymes, topoisomerase inhibitors, biological response modifiers, anti-hormones and anti-androgens.

12. The method of claim 11, wherein the chemotherapeutic agent is doxorubicin.

13. The method of claim 10, wherein the antibiotic agent is selected form the group consisting of penicillins, cephalosporins, polymixins, rifamycins, lipiarmycins, quinolones, sulfonamides, aminoglycosides, macrolides, and tetracyclines cyclic lipopeptides, daptomycin, glycylcyclines, tigecycline, oxazolidinones, linezolid, lipiarmycins, fidaxomicin, or any combination thereof.

14. The method of claim 10, wherein the immunomodulatory agent is selected form the group consisting of an anti-inflammatory agent, an immunosuppressant, an immunostimulatory agent, an antigen, an antibody, or any combination thereof.

15. The method of claim 10, wherein the mitogenic agent is a growth hormone, a cytokine, a chemokine, or any combination thereof.

16. The method of claim 10, wherein the regulator of an ECM is a protease.

17. The method of claim 10, wherein the enzyme is a blood coagulation factor.

18. The method according to claim 1, wherein the subject is susceptible to an adverse side effect of the therapeutic agent when the therapeutic agent is systemically administered.

19. The method according to claim 1, wherein the therapeutic agent is characterized by a certain side effect when administered systemically.

20. The method according to claim 1, wherein the amount effective to treat the condition is less than an amount required to achieve an equivalent efficacy when administered systemically.

21. The method according to claim 1, wherein the amount effective to treat the condition is an amount required to achieve an equivalent efficacy when administered systemically but causes fewer side effects as compared to systemic administration.

22. The method according to claim 1, wherein the amount effective to treat the condition is an amount required to achieve an equivalent efficacy when administered systemically but causes a lesser degree of a side effect as compared to systemic administration.

23. The method according to claim 1, wherein the silk fibroin matrix comprises a reflective element.

24. The method of claim 23, wherein the reflective element comprises a microprism.

25. The method according to claim 1, wherein the silk fibroin matrix further comprises a plurality of nanoparticles.

26. The method of claim 25, wherein the nanoparticle is a thermogenic element.

27. The method of claim 25, wherein a plurality of nanoparticles comprise gold nanoparticles (GNPs).

28. The method according to claim 1, wherein the composition is positioned about 1-60 mm below the surface of the subject's skin.

29. The method according to claim 1, further comprising a step of measuring change in at least one optical property of the silk fibroin delivery matrix over time, wherein the change corresponds to an amount of the therapeutic agent delivered from the silk fibroin delivery matrix.

30. The method according to claim 1, wherein the therapeutic agent is stabilized in the silk fibroin matrix.


SEQUENCE LISTING (22 sequences)

SEQ ID NO: 1 - protein, 90 aa, Artificial sequence
  Synthetic polypeptide
GAGAGSGAGAGSGAGAGSGAGAGSGAGAGSGAGAGSGAGAGSGAGAGSGAGAGSGAGAGSGAGAGSGAGAGSGAGAGSGAGAGSGAGAGS

SEQ ID NO: 2 - protein, 30 aa, Artificial sequence
  Synthetic polypeptide
GXGXGXGXGXGXGXGXGXGXGXGXGXGXGX

SEQ ID NO: 3 - protein, 4 aa, Bombyx mori
GAAS

SEQ ID NO: 4 - protein, 15 aa, Artificial sequence
  Synthetic polypeptide
SSAAAAAAAAAAAAA

SEQ ID NO: 5 - protein, 8 aa, Artificial sequence
  Synthetic polypeptide
GXXXXGGX

SEQ ID NO: 6 - protein, 4 aa, Artificial sequence
  Synthetic polypeptide
GGGX

SEQ ID NO: 7 - protein, 12 aa, Artificial sequence
  Synthetic polypeptide
SSAAAASSAAAA

SEQ ID NO: 8 - protein, 6 aa, Galleria mellonella
GLGGLG

SEQ ID NO: 9 - protein, 6 aa, Artificial sequence
  Synthetic polypeptide
GXGGXG

SEQ ID NO: 10 - protein, 21 aa, Artificial sequence
  Synthetic polypeptide
GPGGXGPGGXGPGGXGPGGXY

SEQ ID NO: 11 - protein, 5 aa, Argiope trifasciata
GRGGA

SEQ ID NO: 12 - protein, 13 aa, Artificial sequence
  Synthetic polypeptide
GAGAAAAAAAGGA

SEQ ID NO: 13 - protein, 8 aa, Artificial sequence
  Synthetic polypeptide
GGXGXGXX

SEQ ID NO: 14 - protein, 16 aa, Bombyx mori
TGSSGFGPYVNGGYSG

SEQ ID NO: 15 - protein, 8 aa, Bombyx mandarina
YEYAWSSE

SEQ ID NO: 16 - protein, 7 aa, Antheraea mylitta
SDFGTGS

SEQ ID NO: 17 - protein, 7 aa, Antheraea yamamai
RRAGYDR

SEQ ID NO: 18 - protein, 8 aa, Galleria mellonella
EVIVIDDR

SEQ ID NO: 19 - protein, 18 aa, Nephila madascariensis
TTIIEDLDITIDGADGPI

SEQ ID NO: 20 - protein, 8 aa, Major ampullata
TISEELTI

SEQ ID NO: 21 - protein, 6 aa, Artificial sequence
  Synthetic polypeptide
GSGAGA

SEQ ID NO: 22 - protein, 5 aa, Artificial sequence
  Synthetic polypeptide
GPGXX